(19) United States
(12) Patent Application Publication
(10) Pub. No.: US 2003/0233124 A1
(43) Pub. Date: Dec. 18, 2003

(54) Methods of treating disorders by altering ion flux across cell membranes with electric fields

(75) Inventors: Akikuni Hara, Tokyo (JP); Hiroyuki Hara, Tokyo (JP); Naoyoshi Suzuki, Tokyo (JP); Shinji Harakawa, Tokyo (JP); Nobuo Uenaka, Tokyo (JP); David E. Martin, Charlottesville, VA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(73) Assignee: Hajuku Institute for Health Science Co., LTD.

(21) Appl. No.: 10/417,142

(22) Filed: Apr. 17, 2003

Related U.S. Application Data
(63) Continuation-in-part of application No. 10/017,105, filed on Dec. 14, 2001.
(60) Provisional application No. 60/433,766, filed on Dec. 17, 2002.
(60) Provisional application No. 60/399,249, filed on Jul. 30, 2002.

(30) Foreign Application Priority Data
Dec. 18, 2000 (JP) 2000-404337

(51) Int. Cl. A61N 1/18

(52) U.S. Cl. 607/3

(57) ABSTRACT

The invention relates to methods and devices for treating disorders with electric current or electric field therapy. The invention uses applied electric current or current induced by an external electric field to manipulate movement of ions across cell membranes and to alter ionic concentrations. The invention is useful, for example, for treating hyperproliferative and cardiovascular disorders and for ameliorating the effects of stress.

### CROSS-REFERENCE TO RELATED APPLICATIONS

This application is a continuation-in-part of U.S. application Ser. No. 10/017,105, filed Dec. 14, 2001, published Dec. 5, 2002. This application also claims the benefit of U.S. Provisional Application No. 60/433,766, filed Dec. 17, 2002, and U.S. Provisional Application No. 60/399,249, filed Jul. 30, 2002. All of the foregoing applications are herein incorporated by reference in their entireties.

### BACKGROUND OF THE INVENTION

Various electrical therapy devices are known. Typically, the electrodes of a device contact the patient, in which case the electrical therapy device employs applied current and may be referred to as an electric current therapy device. Examples include TENS or PENS (Ghoname, E. A., et al., Anesth. Analg., 88:841-46 (1999); Lee, R. C., et al., J Burn Care Rehabil., 14:319-335 (1993)).

If the electrodes do not contact the patient, the electrical therapy device induces current in the patient by means of an external electric field (hereinafter “EF”), and may be referred to as an electric field or electric potential therapy device. EF produces surface charges on all conductive bodies within it, including animal or human bodies. When EF is applied, positive and negative charges will appear on opposite sides of a body. As the field alternates, the charges will alternate in position, resulting in alternating current within the body. (See Hara, H., et al., Niigata Med., 75:265-73 (1961)).

In 1972, Japan's Ministry of Health and Welfare approved an electrical stimulation device (Approval No. 14700BZZ00904). In 1978, the USFDA approved electrical stimulation to treat bone disease. The therapeutic literature, however, reports a wide variety of biological responses to electrical stimulation. For example, external sinusoidal alternating electric fields (ac EF) have been shown to alter, among other things, cellular morphology, protein synthesis in fibroblasts, redistribution of integral membrane proteins, DNA synthesis in cartilage cells, intracellular calcium ion concentration, microfilament structure in human hepatoma cells, and electrolyte levels in blood (Kim, Y. V., et al., Bioelectromagnetics, 19:366-376 (1998); Cho, M. R., et al., FASEB J., 13:677-682 (1999); Hara, H., Niigata Med., 75:265-73 (1961)). Some researchers believe that many of the observed effects do not result from EF directly, but are secondary effects of the influence of EF on primary cellular structures such as membrane-receptor complexes and ion-transport channels.

Although the biological effects of induced current have been studied for the last 25 years, most of the studies were motivated by the safety of persons exposed to intense electrical or magnetic fields from high transmission power lines and related electrical devices. Utility-company workers, for example, are routinely exposed to electric fields of 50-500 kV/m and magnetic fields as high as 5 G, and the general public is commonly exposed to electric fields of 1-10 kV/m and magnetic fields up to 2 G (Portier, C. J. & Wolfe, M. S. (eds.) Assessment of Health Effects from Exposure to Power-line Frequency Electric and Magnetic Fields, NIEHS Publ. No. 98-3981 (National Institute of Environmental Health Sciences, 1998)). The prior art lacks sufficient studies of the effects of relatively low voltage and weak electric fields. In addition, conventional EF therapy devices employ high voltages and do not account for differences in EF intensity across disparate areas of the body's morphology.

In short, as noted by Sporer in U.S. Pat. No. 5,387,231, “[t]he prior art has not contemplated the proper, effective combination of electrical parameters for truly effective electrotherapy. Prior art apparatus generally has operated at very high voltages or very high currents, both of which can have a diathermy effect on the tissue being treated. In many cases, the prior art may mention one or another of the various electrical parameters, but fails to consider the importance of other parameters.”

Since the prior art exhibits disparate biological responses and relies on imprecise measurement and focuses on the effects of high voltage and high current, there remains a need to identify specific parameters for electrical therapy, particularly electrical therapy that employs relatively low voltage and current.

### SUMMARY OF THE INVENTION

The inventors have determined the parameter values of EF and applied current that successfully treat specific disorders. Such parameters include, for example, frequency (in Hertz), voltage (in volts), induced current density (in mA/m²), applied current density (in mA/m²), duration of individual continuous periods of exposure (in minutes, hours, and days), and overall duration of exposure (either as one continuous period of exposure or the sum total of multiple continuous periods of exposure).

As used herein, “mean” applied current density and “mean” induced current density refer to the average current per unit area generated over the cell membranes of at least one organism of interest, for example, a human, animal, plant, or a portion thereof, or cells thereof. For example, if the organism of interest is a human and the portion of interest is the human's entire hand, the mean current density is the average value for the entire hand, that is, the mean current density is the sum of the current densities in each part of the hand divided by the sum of their areas. Specific formulas and techniques, described later herein, are used to estimate the mean applied current density and mean induced current density. Unless explicitly stated otherwise, the term “organism” encompasses both humans and other types of organisms.

One embodiment of the present invention relies on applied electric current. Preferably, the applied current density is in the range of about 10 to about 2,000 mA/m².

Another embodiment of the invention relies on particularly low amounts of induced current to control the movement of ions across cell membranes. For treating disorders that cause or are caused by an abnormal concentration of ions in cells of an organism, this induced current embodiment includes subjecting the organism to an external electric field that generates a mean (average) induced current density over the membranes of the cells of about 0.001 mA/m² to about 15 mA/m², preferably about 0.001 mA/m² to about 10 mA/m², more preferably about 0.01 mA/m² to about 2 mA/m². In preferred embodiments, the external electric field (E) is measured in terms of the expression E=I/εoωS, in which S is a section of the electric field measurement sensor, εo is an induction rate in a vacuum, I is a current, ω is 2πf, and f is frequency. It is also preferable to measure the induced current (J) in terms of the expression J=I/B, in which I is a measured current, B is a circle area expressed as B=A²/4π, A is a circumference expressed as A=2πr, and r is a radius. In additional preferred embodiments of the invention, the induced current density is generated over the cell membranes for a continuous period of about 10 minutes to about 240 minutes. In reapplication, the mean induced current density is preferably generated for additional continuous periods of about 30 minutes to about 90 minutes, preferably resulting in an overall exposure duration of less than about 1,500 minutes.

Both the applied current and induced current embodiments of the invention may be applied to an entire body or to just a portion thereof. A portion thereof may include a limb, an organ, certain bodily tissue, a region of a body such as the trunk, bodily systems, or subsections thereof. A trained individual can determine whether a particular disorder warrants the application of the invention to an entire body or a portion thereof.

The invention may further comprise providing to the organism a calcium supplement, a vitamin D supplement, a lectin supplement, or a combination of these supplements. Preferably, the lectin supplement comprises concanavalin A or wheat germ agglutinin.

In preferred embodiments, the invention alters the flux of or otherwise affects calcium or other cations or polyvalent cations, including cationic electrolytes and proteins in extracellular fluids that play critical roles in activating the electro-sensitive calcium receptor (CaR) associated with Ca++0 uptake.

An alternative embodiment of the invention concerns a device used for the EF therapy. A preferred EF therapy device is an electric field therapy apparatus comprising: a main electrode and an opposed electrode; a voltage generator for applying a voltage to the electrodes; an induced current generator that controls the external electric field by varying the voltage or the distance between the opposed electrode and the organism or portion thereof; and a power source for driving the voltage generator. Preferably, the voltage generator has a booster coil and is grounded at the mid point or at one end of the booster coil.

In a more preferred EF therapy device of the invention, which has a main electrode and an opposed electrode, the opposed electrode is placed near the head, shoulders, abdomen, waist or hips of a human body and the distance between the opposed electrode and the surface of the human subject's trunk area is about 1 to 25 cm, more preferably about 1 to 15 cm. In alternative forms, the opposed electrode is the ceiling, wall, floor, furniture or other objects or surfaces in the room.

Another alternative embodiment concerns determining optimal parameters for the EF or applied current therapy. A preferred method of determining optimal parameters for EF therapy includes the following steps: (i) identifying a desired biological response to elicit in a living organism; (ii) selecting or measuring a mean induced current density over membranes of cells in the organism or in a tissue sample or culture derived from the organism; (iii) selecting or measuring an external electric field that generates the selected or measured induced current density at a particular distance from the organism, sample or culture; (iv) selecting or measuring a continuous period of time to generate the selected or measured induced current density over the membranes; (v) applying the selected or measured electric field to the organism, sample or culture to generate the selected or measured induced current density over the cell membranes for the selected or measured continuous period of time; (vi) determining the extent to which the desired biological response occurs; (vii) optionally repeating any of steps (ii) through (vi); and/or (viii) identifying the values for the selected or measured induced current density, for the selected or measured external electric field, or for the selected or measured continuous period of time that optimally elicit the desired biological response. With regard to this embodiment, the term “measuring” encompasses instances in which the experimenter does not consciously, deliberately or initially pre-select the parameter value. For example, the term measuring encompasses cases where an EF device generates a random or initially unknown amount of mean induced current density and thereafter the researcher directly or indirectly determines what that amount is.

The invention is further illustrated by the following figures and detailed descriptions.

### BRIEF DESCRIPTION OF THE DRAWINGS

FIG. 1 shows a field exposure dish in an EF exposure system.

FIG. 2 displays the percentage of viable cells following EF exposure.

FIG. 3 shows a significant increase in the number of [Ca²⁺]_{c}-high cells in both EF-exposed and unexposed cell suspensions containing 12.5 μg/ml Con-A.

FIGS. 4A and 4B summarize the results of EF-exposed cell cultures containing different concentrations of Con-A, with and without 1 mM of CaCl₂.

FIG. 5 shows significant increases in [Ca²⁺]_{c}-high cells in both EF-exposed and unexposed cells containing phytohemaglutinin (PHA).

FIG. 6 shows a significant increase in [Ca²⁺]_{c}-high cells of either EF-exposed or unexposed cells when supplemented with 3.125-12.5 μg/ml of Con-A, when compared to those cells stimulated with 0.025 μg/ml of Con-A.

FIG. 7 demonstrates that the ConA-induced concentration of calcium ion increased in the splenocyte cells.

FIG. 8 displays the time course change of DiBAC dye intensity in BALB 3T3 mouse embryo cells stimulated with a final concentration of 0.4 μM A23187.

FIG. 9 shows the effects on membrane potential in BALB 3T3 of an electric field (EF) at 100 Hz that generates a current density of approximately 200 μA/cm².

FIG. 10 also shows the effects on membrane potential in BALB 3T3 of an electric field (EF) at 100 Hz that generates a current density of approximately 200 μA/cm².

FIG. 11 displays the effect of stress on plasma adrenocorticotropic hormone (hereinafter “ACTH”) levels.

FIGS. 12A and 12B show the effect of exposure to EF on plasma ACTH level in normal (A) and ovariectomized rats (B).

FIG. 13 shows the effect of EF exposure on plasma ACTH levels in normal rats (n=6).

FIGS. 14A and 14B show the effect of EF exposure on restraint-induced plasma glucose level changes on normal (A) and ovariectomized rats (B).

FIGS. 15A and 15B show the effect of EF exposure on restraint-induced plasma lactate levels in normal (A) and ovariectomized rats (B).

FIG. 16 shows the effect of EF exposure on restraint-induced plasma pyruvate levels in ovariectomized rats.

FIG. 17 shows the effect of EF exposure on restraint-induced white blood cell (WBC) counts in ovariectomized rats.

FIG. 18 demonstrates a conceptual contour of an electric field generated using an EF therapy device, in this case a BioniTron Chair from Hakuju Institute for Health Science.

FIG. 19 is a schematic view of a preferred EF therapy apparatus of the invention.

FIGS. 20A and 20B show another preferred EF therapy apparatus.

FIGS. 21A and 21B show another preferred EF therapy apparatus.

FIG. 22 is a diagram showing a preferred electric configuration of the EF therapy apparatus.

FIG. 23A is a front view of a simulated human body, FIG. 23B is a perspective view, and FIG. 23C is a view showing an EF measurement sensor attached to the neck of the body.

FIG. 24 shows a device for measuring the induced current generated by the EF therapy apparatus.

FIG. 25 shows the relationship between an applied voltage and an induced current.

FIG. 26 shows the relationship between the position of a head electrode and current induced in the neck.

FIG. 27 demonstrates induced current densities (mA/m²) at various locations in an ungrounded human subject.

FIG. 28 shows the palliative effect of EF exposure on various symptoms in humans.

### DETAILED DESCRIPTION OF THE INVENTION

A. Method of Modulating Ion Flux Across Cell Membranes

An ionic imbalance may result from a disorder or condition or may be a side effect of a medical treatment or supplement. The invention alters ion flux across cell membranes by generating an electric current over the membranes. The invention also influences components of the cell membrane such as its transmembrane proteins. The invention can restore or equilibrate cellular ionic homeostasis or alter the membrane potential of cell membranes. Thus, the invention is useful for the prevention or treatment of disorders associated with cellular and extracellular ion concentrations, such as concentrations of calcium (Ca²⁺), magnesium (Mg²⁺), sodium (Na⁺), potassium (K⁺), and chlorine (Cl⁻).

For treating disorders associated with serum calcium concentrations, the mean induced current density generated over the cell membranes is preferably about 0.3 mA/m² to about 0.6 mA/m², more preferably about 0.4 mA/m² to about 0.5 mA/m², most preferably about 0.42 mA/m². Using applied current to treat a disorder associated with serum calcium concentration, the mean applied current density is preferably about 60 mA/m² to about 2,000 mA/m² and the mean applied current density is generated over the cell membranes for a continuous period of about 1 minute to about 20 minutes, more preferably about 2 to about 10 minutes.

Tissues for which the methods of the invention may be used include, for example, musculo-skeletal tissues, tissues of the central and peripheral nervous system, gastrointestinal system tissues, reproductive system tissues (both male and female), pulmonary system tissues, cardiovascular system tissues, endocrine system tissues, immune system tissues, lymphatic system tissues, and urogenital system tissues.

Biological membranes of eukaryotic cells, such as the plasma membrane, are selectively permeable to these ions. The selective permeability allows for the establishment of a membrane potential across the membrane. The cell harnesses the membrane potential for the transport of molecules across membranes. Many of the ions associated with the generation of a membrane potential perform vital functions. For example, a threshold concentration of calcium ions in muscle cells initiates contraction. In exocrine cells of the pancreatic system, a threshold concentration of calcium ions triggers the secretion of digestive enzymes. Similarly, various concentrations of sodium and potassium ions are essential to the conductance of electric impulses through nerve axons.

A broad family of proteins called voltage-gated ion channels maintains ion concentrations and membrane potentials. Voltage-gated ion channels are trans-membrane proteins containing ion-selective pores that allow ions to pass across the biological membrane, depending upon the conformational state of the channel. The conformational state of the channel is influenced by a voltage-sensitive portion that contains charged amino acids that react to the membrane potential. The channel is either conducting (open/activated) or nonconducting (closed/nonactivated).

Due to the association of particular ions (i.e., Ca²⁺) with cardiovascular health, the invention is useful for the prevention or treatment of cardiovascular disorders. These include, for example, cardiomyopathy, dilated congestive cardiomyopathy, hypertrophic cardiomyopathy, angina, variant angina, unstable angina, atherosclerosis, aneurysms, abdominal aortic aneurysms, peripheral arterial disease, blood pressure disorders such as low blood pressure and high blood pressure, orthostatic hypotension, chronic pericarditis, arrhythmias, atrial fibrillation and flutter, heart disease, left ventricular hypertrophy, right ventricular hypertrophy, tachycardia, atrial tachycardia, ventricular tachycardia, and hypertension.

The invention is also useful for the prevention or treatment of disorders of the blood. These include, but are not limited to, hyponatremia, hypernatremia, hypokalemia, hyperkalemia, hypocalcemia, hypercalcemia, hypophosphatemia, hyperphosphatemia, hypomagnesemia, and hypermagnesemia, as well as blood-glucose regulatory disorders such as diabetes, adult-onset diabetes, and juvenile diabetes.

In one embodiment of the invention, a lectin is co-applied with the EF to enhance Ca²⁺ flux across the cell membrane. Lectins useful for the invention include, for example, concanavalin A (ConA) and wheat germ agglutinin. In another embodiment, the ion flux generated by the invention is generated concurrently with a calcium supplementation. In another embodiment, the ion flux generated by the invention is generated concurrently with a vitamin D supplementation or with both a calcium supplementation and a vitamin D supplementation. Vitamin D supplements of the invention include, for example, vitamin D₂ (ergocalciferol) and vitamin D₃ (cholecalciferol). Similarly, the methods of the invention can be administered in conjunction with a supplemental light source that is administered to the surface of a biological sample or patient. The light source may emit a wavelength in the range of from about 225 nanometers to about 700 nanometers. In one embodiment of the invention, the light source co-applied with the methods of the invention emits a wavelength in the range of from about 230 nanometers to about 313 nanometers.

In an additional embodiment of the invention, another molecule may transfer across a cell membrane concurrently with an ion flux generated by the invention. The additional molecule that may transfer concurrently with the ion flux may be naturally produced by the body, or alternatively may be provided by way of supplementation (e.g., via a vitamin, etc.). Cellular glucose uptake, for example, may be enhanced by calcium ion flux across a cell membrane. Additional molecules that may be transferred across a cell membrane concurrently with an ion flux generated by the invention include neutraceuticals (e.g., a nutritional supplement designed and dosed to aid in the prevention or treatment of a disorder and/or condition). Additionally, the methods of the invention may be used in conjunction with hyperalimentation treatment (e.g., the administration of nutrients beyond normal requirements for the treatment of disorders, such as for example, coma or severe burns or gastrointestinal disorders).

### EXAMPLE 1

60 Hz Electric Field Upregulates Cytosolic Calcium (Ca²⁺) Level in Mouse Splenocytes Stimulated by Lectins

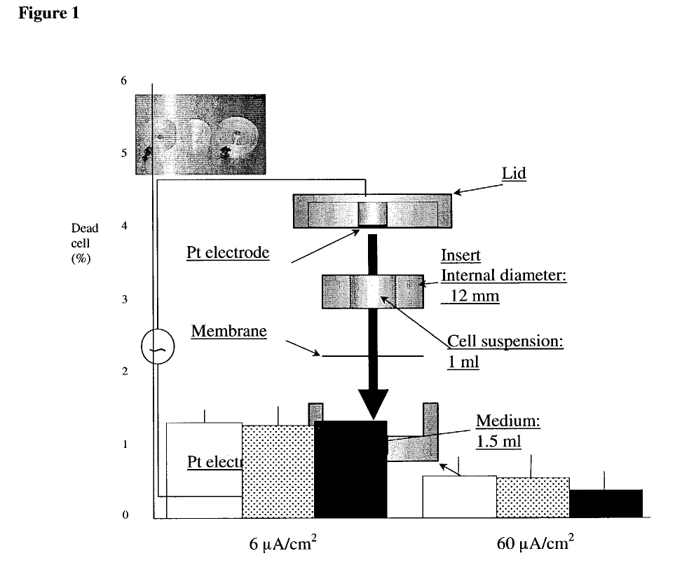

The EF exposure system utilized for this experiment was composed of four parts: the field exposure dish made of polycarbonate; the function generator (SG-4101, IWATSU Co. Ltd., Tokyo, Japan); the digital multi-meter (VOAC-7411 IWATSU, Tokyo, Japan); and the controller (Hakuju Co. Ltd., Tokyo, Japan). FIG. 1 shows a field exposure dish in an EF exposure system. The field exposure dish is composed of a lid, a dish and a doughnut-shaped insert (internal diameter: 12 mm). An EF was generated between the two round-shape platinum electrodes (the cell culture space) by the function generator, and was finely adjusted by using the controller and the digital multi-meter. The field strength of 60 Hz electric field was determined by measuring a current density within the cell culture space of the field exposure dish.

The current density was calculated by the expression: Current density=I/S, where “I” is the supplied current (μA), and S is the area (cm²) of the cell culture space (0.36π). Thus, the current density can be calculated by: Current density=0.885I [μA/cm²]

Prior to the EF exposure, approximately 1.5 ml of the assay buffer (137 mM NaCl, 5 mM KCl, 1 mM Na₂HPO₄, 5 mM glucose, 1 mM CaCl₂, 0.5 mM MgCl₂, 0.1% (w/v) BSA and 10 mM HEPES pH 7.4) was poured into the electrode chamber. In order to avoid contact of the cells and the lower electrode, polycarbonate membrane (Isopore, MILLIPORE, Mass. USA) was placed between the dish and the insert. Approximately 1 ml of the cell suspension was poured into culture well/space and covered with a lid.

Cell Preparation

Female BALB/c mice, 4-7 wk old obtained from CLEA Inc. (Tokyo, Japan) maintained in a conventional animal house equipped with clean air-filtering device were splenectomized under anesthesia, and cell suspensions of splenocytes were prepared. To examine cell viability, the cells were cultivated in Dulbecco's modified Eagle's medium (SIGMA, MO, USA) supplemented with 10% fetal bovine serum (FSB). The cells were maintained in Hank's balanced salt solution (HBSS) (SIGMA, MO, USA) during examination for [Ca²⁺]_{c} which was carried out within 4 hr after cell preparation. Cells were stored at 4 degree C. prior to use.

Determination of the Viability of EF-Exposed Cells

Mouse splenocytes (5×10⁶ cells/ml) were exposed to 60 Hz either at 6 μA/cm² or 60 μA/cm² EF for 30 min and 24 hr, at 37 degrees C. in 5% CO₂. The sham (control) cells were left on the field exposure dish for 30 min and 24 hr but were not exposed to EF. The cell suspensions harvested from the field exposure dish at the end of 30 min, and 24 hr exposure were stained with 2.5 μg/ml propidium iodide for 30 min at 4 degrees C., and percent dead cells were analyzed by flow cytometry.

Cell Preparation for Assay of [Ca²⁺]_{c}-High Cells and Lectins Used

Splenocytes (10⁶ cells/ml) were incubated for 20 min at 37 degrees C. in HBSS containing 2.5 μM fluo-3-acetoxylmethyl (Molecular Probes, USA) [Vandenberghe et al., 1990]. The cell suspension was then diluted 5 times with HBSS containing 1% FBS, incubated for 40 min at 37 degrees C., washed 3 times with assay buffer, and the cells were then suspended in the assay buffer at a concentration of 1×10⁶/ml. Throughout the cell preparation, the cell suspensions were mixed gently.

Considering the reported synergistic interaction between EMF and mitogen (Walleczek and Liburdy, 1990), concanavalin-A (Con-A) (Seikagaku Co., Tokyo, Japan) and phytohemaglutinin (PHA) (SIGMA, MO, USA) were used.

Experimental Design to Determine the Effect of 60 Hz (6 μA/cm²) EF on the Generation of [Ca²⁺]_{c}-High Cells

Taking into account the results of the viability test for exposed murine splenocytes earlier assayed, we chose to use the optimum culture and exposure conditions (60 Hz, 6 μA/cm² EF) in carrying out the following five experiments:

(1) cells suspended in HEPES-buffered saline (BS)+1 mM CaCl₂ were exposed to EF for a total of 40 min, and 12.5 μg/ml of Con-A was added after the first 8 min of exposure. The control groups consisted of EF-unexposed cells containing Con-A, and EF-exposed cells without Con-A. Percent [Ca²⁺]_{c}-high cells was checked at certain exposure points;

(2) cells in HEPES-BS+1 mM CaCl₂ were exposed for a total of 12 min, and different concentrations (1 ng-12.5 μg/ml) of Con-A were added after the first 4 min of exposure. The control group was essentially the same as that of the experimental group but without EF-exposure;

(3) cells in HEPES-BS+1 mM CaCl₂ were exposed for a total of 8 min, and 5 μg/ml of PHA was added after the first 4 min of exposure. The control groups consisted of EF-unexposed cells containing PHA, and EF-exposed cells without PHA;

(4) cells suspended in HEPES-BS without CaCl₂ were exposed for a total of 12 min, and different concentrations (1 ng-5 μg/ml) of Con-A were added after the first 4 min of exposure. The control group was essentially the same as the experimental group but without EF exposure; and

(5) to evaluate the persistent effect of EF exposure, cells suspended in HEPES-BS+1 ml CaCl₂ were exposed for a total of 4 min, after which different concentrations (0.025-12.5 μg/ml) of Con-A were added, and the generation of [Ca²⁺]_{c}-high cells for the next 8 min without EF exposure was monitored with flow cytometry. The control was essentially the same as the experimental group but without any EF-exposure.

Statistical Analysis

Statistical analysis in cell viability was determined using the Student's t test. Data for the effect by exposure of EF in [Ca²⁺]_{c} among groups was analyzed by ANOVA (ANalysis Of VAriance between groups), Student's t test and paired t test. All computations for the statistical analysis were carried out in MS-EXCEL® Japanese Edition (Microsoft Office software: Ver. 9.0.1, Microsoft Japan Inc. Tokyo, Japan).

Results

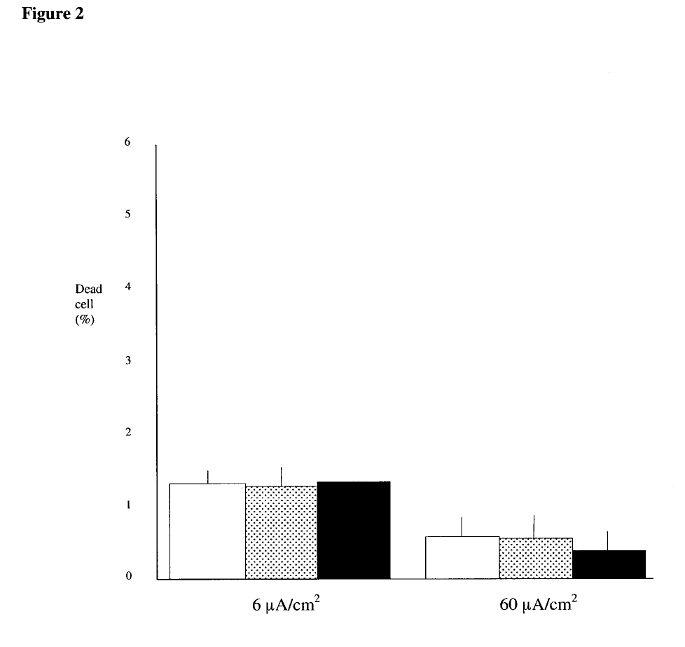

FIG. 2 displays the percentage of viable cells following EF exposure. In all three replicates, more than 98% of the cells were viable after exposure to either 6 μA/cm² or 60 μA/cm².

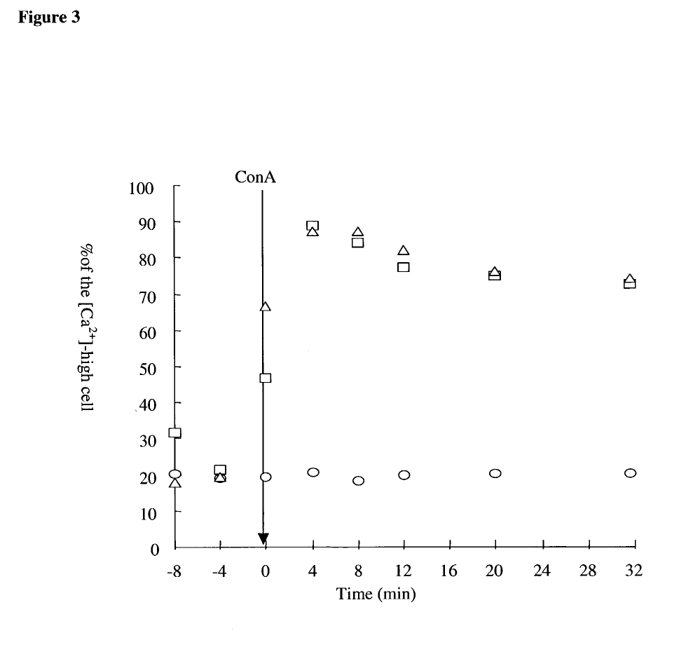

The number of [Ca²⁺]_{c}-high cells increased significantly in both EF-exposed and unexposed cell suspensions containing 12.5 μg/ml Con-A (FIG. 3). In FIG. 3, the circles represent suspensions without Con-A, the triangles represent suspensions with Con-A that were exposed to EF and the squares represent suspensions with Con-A that were not exposed to EF. Those in EF-exposed cell suspension without Con-A remained essentially unchanged. The Con-A-induced response was noted immediately and reached a saturation point within 5-8 minutes after the addition of the mitogen. The differences between EF exposed and unexposed Con-A-induced cells were insignificant (P>0.05).

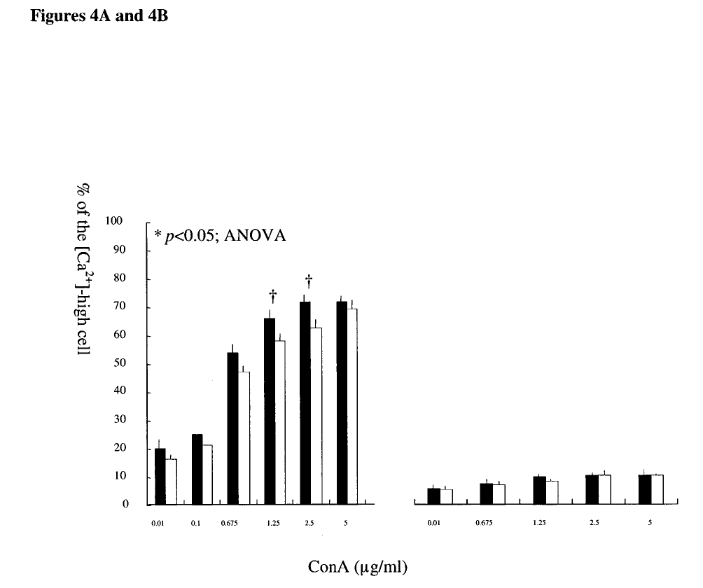

FIGS. 4A and 4B summarize the results of EF-exposed cell cultures containing different concentrations of Con-A, with and without 1 mM of CaCl₂. FIG. 4A shows the results for the cultures with 1 mM of CaCl₂. In FIG. 4A, both the EF-exposed cultures (black bars) and the cultures not exposed to EF (white bars) contain 1 mM of CaCl₂ and contain various concentrations of Con-A (0.01 μg/ml to 5 μg/ml). In the presence of CaCl₂ (FIG. 4A), the EF significantly enhanced the Con-A dependent [Ca²⁺]_{c} (P<0.01: ANOVA). Although the increase in [Ca²⁺]_{c}-high cells was more substantial in the 0.675-5.0 μg/ml Con-A stimulated groups, only the 1.25 μg/ml and 2.5 μg/ml Con-A-induced cells showed significant differences (P<0.05: paired t test). In FIG. 4B, both the EF-exposed cultures (black bars) and the control cultures not exposed to EF (white bars) contain the various concentrations of Con-A but contain no CaCl₂. Con-A-dependent [Ca²⁺]_{c} rise was negligible in the Ca²⁺-free cell condition (FIG. 4B) in both the control and the EF-exposed groups.

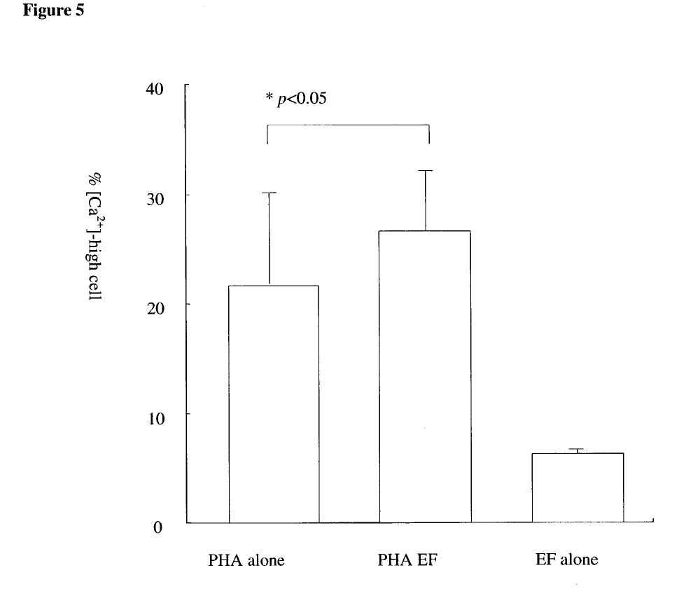

To determine whether the EF-dependent [Ca²⁺]_{c} upregulation was limited to Con-A, PHA-stimulated cells were also assayed. Both EF-exposed and unexposed cells containing PHA registered significant increases in [Ca²⁺]_{c}-high cells (FIG. 5). The increase in EF-exposed cells however was significant (P<0.05: paired t test) relative to the unexposed group.

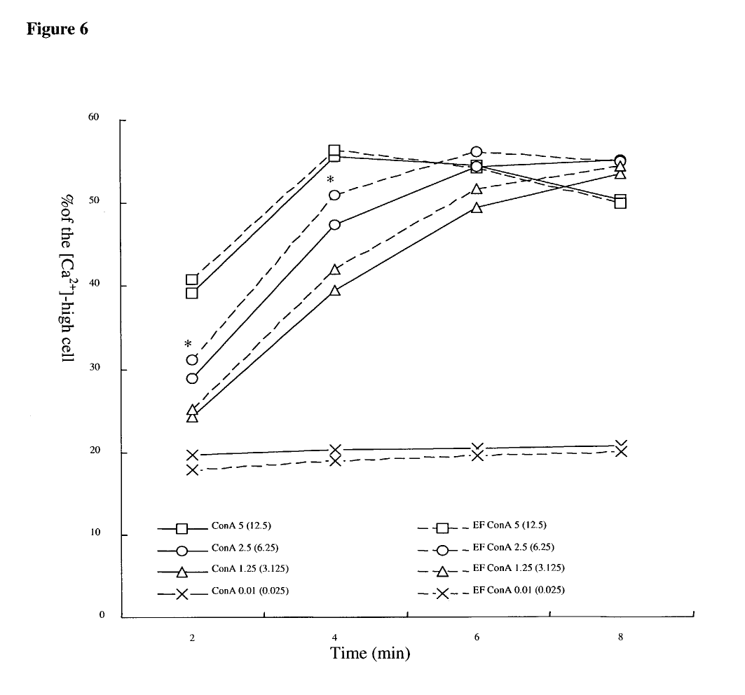

The addition of 3.125-12.5 μg/ml of Con-A to cell suspensions either unexposed or earlier exposed to EF for 4 min showed significant increase in [Ca²⁺]_{c}-high cells compared to those cells stimulated with 0.025 μg/ml of Con-A (FIG. 6). Cells stimulated with 3.125 and 6.25 μg/ml Con-A exhibited sustained increase in [Ca²⁺]_{c}-high cells which leveled off at about 8 min post-Con-A stimulation, while cell cultures stimulated with higher concentration of Con-A (12.5 μg/ml) showed a decline in [Ca²⁺]_{c}-high cells approximately 4 min post-Con-A stimulation. The enhancing effect of EF exposure was significantly demonstrable at 2-4 min only in the presence of 6.25 μg/ml of Con-A (P<0.05: paired t test).

### EXAMPLE 2

Effects of Low Frequency Electric Fields on Vasoactive Substance-Induced Intracellular Calcium (Ca²⁺) Responses in Human Vascular Endothelial Cells.

To evaluate the effects of EF on human vascular endothelial cells (hereinafter HUVEC), intracellular calcium levels were examined in HUVEC stimulated with ATP and histamine. To evaluate the effects of EF on HUVEC, HUVEC were exposed to a 50 Hz (30,000 V/m) EF, 3,000 volts. It is estimated that the EF induced current density on HUVEC was 0.42 mA/m2. HUVEC were exposed to these test parameters for 24 hrs.

After exposure, the cytoplasmic free Ca²⁺ concentration was determined by fluo3 flow cytometry. A change in fluo3 image intensity was confirmed with real-exposure confocal laser microscopy. The results demonstrate that EF increased the concentration of calcium in HUVEC.

B. Method of Treating Proliferative Cell Disorders

For treating proliferative cell disorders, particularly those involving differentiated fibroblast cells, the mean induced current density generated over the cell membranes is preferably about 0.1 mA/m² to about 2 mA/m², more preferably about 0.2 mA/m² to about 1.2 mA/m², and still more preferably about 0.29 mA/m² to about 1.12 mA/m². With applied current, the mean applied current density generated over the cell membranes is preferably about 10 mA/m² to about 100 mA/M².

Fibroblasts are a cell type derived from embryonic mesoderm tissue. Fibroblasts are capable of in vitro culturing, and secrete matrix proteins such as laminin, fibronectin, and collagen. Cultured fibroblasts are not generally as differentiated as tissue fibroblasts. With the proper stimulation, however, fibroblasts have the capability to differentiate into many types of cells, such as for example, adipose cells, connective tissue cells, muscle cells, collagen fibers, etc.

Given that fibroblasts are capable of differentiation into numerous cell types associated with connective tissues and the musculoskeletal system, methods of controlling the growth of undifferentiated fibroblast cells in vivo or in vitro are useful in controlling the growth of differentiated cells derived from fibroblasts. For example, hyperproliferative disorders of musculoskeletal system tissues may be controlled or prevented by methods that prevent the growth of fibroblast cells. We determined that generation over cell membranes of an applied current density of about 10, 50 or 100 mA/m² for a duration of about 24 hours/day for at least about 7 days inhibits growth of cultured fibroblast cells in a current density-dependent manner.

Hyperproliferative disorders include, for example, neoplasms associated with connective and musculoskeletal system tissues, such as fibrosarcoma, rhabdomyosarcoma, myxosarcoma, chondrosarcoma, osteogenic sarcoma, chordoma, and liposarcoma. Additional hyperproliferative disorders that can be prevented, ameliorated or treated using the invention methods include, for example, progression and/or metastases of malignancies such as neoplasms located in the abdomen, bone, brain, breast, colon, digestive system, endocrine glands (adrenal, parathyroid, pituitary, testicles, ovary, thymus, thyroid), eye, head and neck, liver, lymphatic system, nervous system (central and peripheral), pancreas, pelvis, peritoneum, skin, soft tissue, spleen, thorax, and urogenital tract, leukemias (including acute promyelocytic, acute lymphocytic leukemia, acute myelocytic leukemia, myeloblastic, promyelocytic, myelomonocytic, monocytic, erythroleukemia), lymphomas (including Hodgkins and non-Hodgkins lymphomas), multiple myeloma, colon carcinoma, prostate cancer, lung cancer, small cell lung carcinoma, bronchogenic carcinoma, testicular cancer, cervical cancer, ovarian cancer, breast cancer, angiosarcoma, lymphangiosarcoma, endotheliosarcoma, lymphangioendotheliosarcoma, synovioma, mesothelioma, Ewing's sarcoma, leiomyosarcoma, squamous cell carcinoma, basal cell carcinoma, pancreatic cancer, renal cell carcinoma, Wilm's tumor, hepatoma, bile duct carcinoma, adenocarcinoma, epithelial carcinoma, melanoma, sweat gland carcinoma, sebaceous gland carcinoma, papillary carcinoma, papillary adenocarcinoma, glioma, astrocytoma, medulloblastoma, craniopharyngioma, ependymoma, pinealoma, emangioblastoma, acoustic neuroma, oligodendroglioma, menangioma, neuroblastoma, retinoblastoma, bladder carcinoma, embryonal carcinoma, cystadenocarcinoma, medullary carcinoma, choriocarcinoma, and seminoma.

### EXAMPLE 3

Effects of EF Exposure on Ca²⁺ Concentration in Murine Splenocytes and 3T3/A31 Fibroblast Cells

Effect on Murine Splenocytes

In order to determine the effect of EF on calcium ion concentration in murine splenocytes, specific EF field exposures of 60 Hz were applied to murine splenocytes. Mice were splenectomized under anesthesia. In a 60 mm dish, the spleen was injected with PBS (phosphate buffered saline including 0.083% NH₄Cl). The cells were re-suspended and maintained in Hank's balanced salt solution (HBSS) (SIGMA, Mo., USA), during examination for [Ca²⁺]_{c}, which was carried out within 4 hours after cell preparation. Cells were stored at 4° C. prior to use.

The application of a 60 Hz EF to splenocyte cells created applied current densities of 6, 20, 60, and 200 μA/cm². Splenocyte cells were exposed to these conditions for 4 minutes, after which exposure the splenocyte samples were stimulated with Concanavalin A (ConA). Following stimulation of splenocytes with ConA, cytoplasmic free Ca²⁺ concentration was determined by fluo3 flow cytometry.

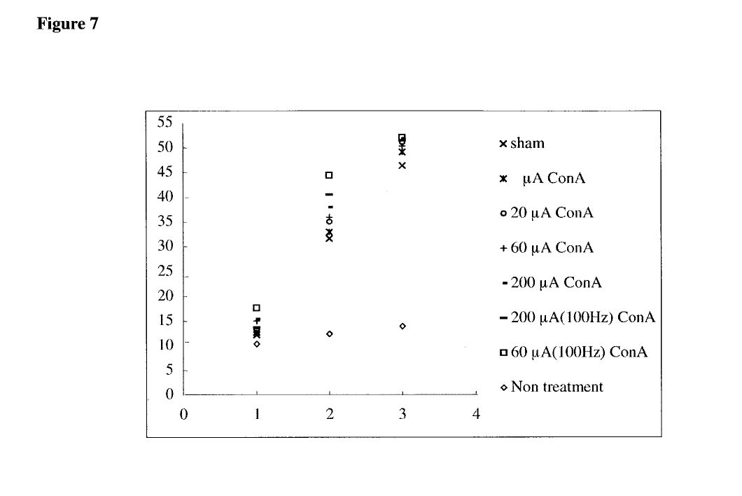

The experiment demonstrates that the ConA increased calcium concentration in the splenocyte cells. The calcium ion concentration increased with an EF that applied 6-200 μA/cm². More importantly, the increase in calcium ion concentration was dependent on current density (See FIG. 7, in which the Y-axis shows calcium concentration and x-axis shows time in minutes).

Effect on BALB 3T3

In order to determine the effect of EF on calcium ion concentration in murine 3T3/A31 fibroblast cells, the 3T3 cells were subjected to an EF at 60 Hz. 3T3 cell lines were obtained from the cell bank of the Japanese National Research Center for Protozoan Disease and grown at 37° C. in DMEM including 5% FCS and 10 mM HEPES.

The EF generated an applied current density over the cells of 200 μA/cm². After 2 minutes of exposure, the cytoplasmic free Ca²⁺ concentration was determined by fluo3 flow cytometry, which showed that the calcium concentration increased in the cells. A change in fluo3 image intensity was confirmed with confocal laser microscopy.

### EXAMPLE 4

Effects of Calcium Ionophore and EF on Membrane Potential in BALB 3T3

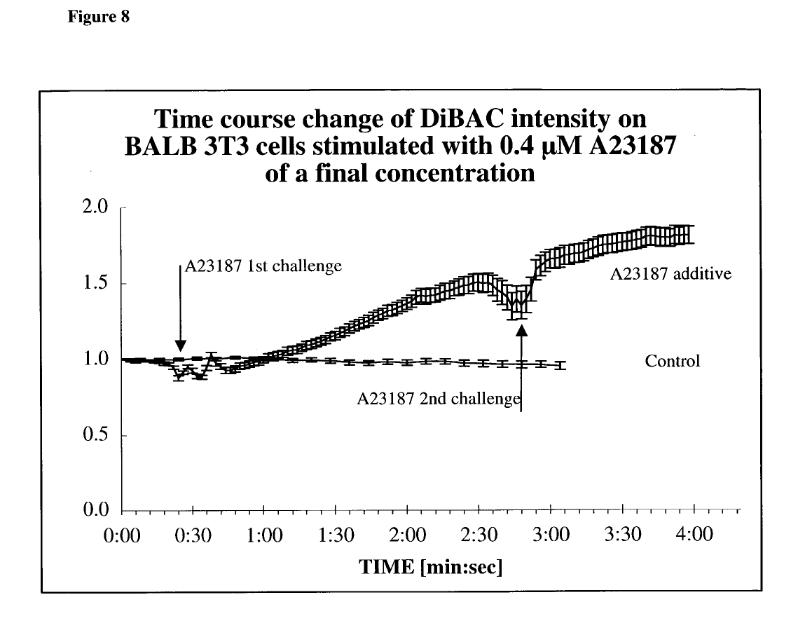

FIG. 8 shows that calcium ionophore alters the membrane potential of murine BALB 3T3/A31 fibroblast/embryo cells. FIG. 8 displays the time course change of DiBAC intensity in BALB 3T3 cells stimulated with a final concentration of 0.4 mM A23187. A23187 is a monocarboxylic acid extracted from Streptomyces chartreusensis that acts as a mobile-carrier calcium ionophore. DiBAC is a fluorescent dye that enters the cell membrane when the membrane's potential changes. Thus, when the membranes of the BALB 3T3 cells depolarize, the DiBAC enters those membranes thereby increasing the intensity of the DiBAC signal (Y-axis) in the BALB 3T3 cells.

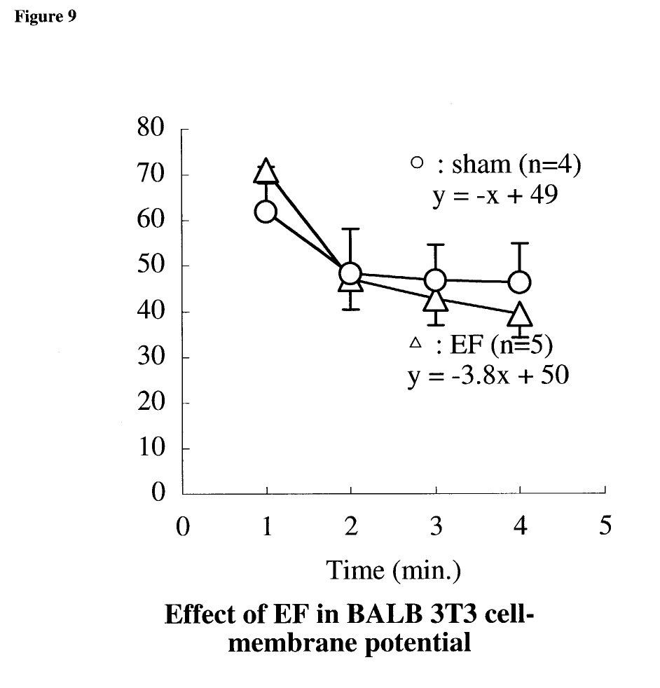

FIG. 9 shows the effects on membrane potential in BALB 3T3 of an electric field (EF) at 100 Hz, which generates a current density of approximately 200 mA/cm2. The changes in membrane potential were measured with flow cytometry. The methodology for the flow cytometry was as follows. Culture in DMEM was supplemented with 5% FCS 10 mM HEPES. It was then de-touched with 0.02% trypsin and 0.025% EDTA. It was then re-suspended in HEPES buffered saline, 137 mM NaCl, 5 mM KCl, 1 mM Na2HPO4, 5 mM glucose, 1 mM CaCl2, 0.5 mM MgCl2, 0.1% (w/v) BSA and 10 mM HEPES pH 7.4. It was then loaded with DiBAC4(3) of a final concentration of 200 nM. It was incubated at 37 degree C. for >5 min. Then the flow cytometry measurements were performed.

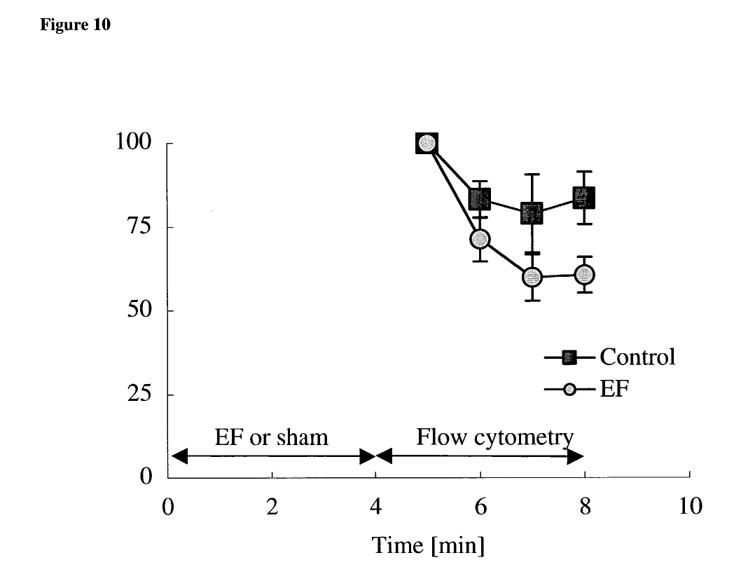

FIG. 10 also shows the effects on membrane potential in BALB 3T3 of an electric field (EF) at 100 Hz that generates a current density of approximately 200 mA/cm2.

### EXAMPLE 5

Extracellular Currents Alter Gap Junction Intercellular Communication in Synovial Fibroblasts

We examined the effect of low-level currents on gap-junction intercellular communication (GJIC) mediated by connexin43 protein. Confluent monolayers of synovial fibroblasts (HIG-82) and neuroblastoma cells (5Y) were exposed in bath solution to 0-75 mA/m² (0-56 mV/m, 60 Hz), and single-channel conductance, cell-membrane current-voltage (I-V) curves, and Ca²⁺ influx were measured using the nystatin double- and single-patch methods. The conductances of the closed and open states of the gap-junction channel in HIG-82 cells were each significantly reduced in cells exposed to 20 mA/m² (by 0.76 pA and 0.39 pA, respectively); no effect occurred on the conductance of the gap-junction channel between 5Y cells. Current densities as low as 10 mA/m² significantly increased Ca²⁺ influx in HIG-82 cells, but had no effect on 5Y cells. The I-V curves of the plasma membranes of both types of cells were independent of 60-Hz currents, 0-75 mA/m², indicating that the effect of the 60-Hz currents on GJIC in HIG-82 cells was not mediated by a change in membrane potential.

The conclusion was that low-level extracellular currents could alter GJIC in synovial cells via a mechanism that does not depend on changes in membrane potential, but may depend on Ca²⁺ influx. The results suggest that GJIC-mediated responses in synovial cells, for example, their secretory responses to pro-inflammatory cytokines, could be antagonized by the application of extracellular low-frequency currents.

C. Method of Reducing Stress

The invention is useful for the prevention or treatment of stress and stress-associated disorders, such as reduced immune-system function, infections, hypertension, atherosclerosis, and insulin-resistance-dyslipidemia syndrome. For treating stress, immunosuppressive disorders and for reducing levels of ACTH or cortisol, the mean induced current density generated over the cell membranes is preferably about 0.03 mA/m² to about 12 mA/m², more preferably 0.035 mA/m² to about 11.1 mA/m². With applied current, the mean applied current density is preferably about 60 mA/m² to about 600 mA/m².

Stress is associated with numerous health disorders, including hypertension, atherosclerosis, and the insulin-resistance-dyslipidemia syndrome, as well as certain disorders of immune function (Vanitallie T. B., Metabolism, 51:40-5 (2002)). Researchers have observed that stress can influence the normal homeostasis of adrenocortical hormones, such as cortisol and corticosterone. The hormone corticosterone is produced by the adrenal gland, and changes in it are a general indicator of stress. In a report involving mice exposed to electric fields of up to 50 kV/m, 60 Hz, reductions in plasma corticosterone concentrations were observed, but only at the beginning of the exposure period (Hackman, R. M. & Graves, H. B., Behav. Neural Biol. 32:201-213 (1981)). Similarly, Portet and Cabanes reported that when rabbits and rats were exposed to 50 kV/m, 50 Hz, lowered cortisol levels were found in the adrenal gland but not in blood cortisol concentrations (Portet, R. & Cabanes, J., Bioelectromagnetics 9:95-104 (1988)).

ACTH is a peptide expressed by the pituitary gland, and almost exclusively controls the secretion of cortisol. ACTH levels in the body function as a strong indicator of bodily stress levels, primarily because ACTH functions to control the secretion of cortisol (a major anti-inflammatory molecule crucial for stress responses to, for example, traumatic events). Interestingly, researchers have found no increase in ACTH levels after 30-120 days of field exposure (Free, M. J., et al., Bioelectromagnetics 2:105-121 (1981)). In a study where rats were exposed to 100 kV/m, 60 Hz, for 1-3 hours, no changes in plasma ACTH were found (Quinlan, W. J., et al., Bioelectromagnetics 6:381-389 (1985)). When mice were exposed to 10 kV/m, 50 Hz, the serum ACTH concentration was higher than in the controls (deBruyn, L. & deJager, L., Environ. Res. 65:149-160 (1994)). Lipid staining in a region of the adrenal cortex was elevated, but only in the males. The authors concluded that the electric field was a stressor. Altered blood ACTH concentrations were also observed in rats exposed to a 15 kV/m, 60 Hz electric field for 30 days (Marino, A. A., et al., Physiol. Chem. Phys. 9:433-441 (1977)).

In contrast, we have determined that the application of an electric field at particular parameters to test animals results in the reduction of stress-induced ACTH concentrations. For example, the application of a 17,500 V/m electric field (50 Hz), a voltage of 7,000 V, and an induced current density of about 0.035-0.5 mA/m² for a duration of 60 minutes resulted in the reduction of stress-induced serum ACTH-levels in test animals.

### EXAMPLE 6

Effect of a 50 Hz Electric Field in Plasma ACTH, Glucose, Lactate and Pyruvate Levels on Restrained Rats

Electric Field Exposure System

The EF exposure system used in this example was composed of three major parts: a high voltage generator (Healthtron TM, maximum output voltage: 9,000 V; Hakuju Institute for Health Science Co. Ltd., Tokyo, Japan), a constant-voltage power supply (TOKYO SEIDEN, Tokyo, Japan), and EF exposure cages. The exposure cage is composed of a cylindrical plastic cage (φ: 400 mm, height: 400 mm) and two electrodes made of stainless steel (1,200×1,200 mm) placed over and under the cylindrical cage. In order to form the EF (50 Hz; 17,500 V/m) in the cage, stable alternating current (50 Hz; 7,000 V) was applied to the upper electrode.

Experimental Animal

Female, 7 week old Wistar rats, 300-350 g of body weight, were purchased from Charles River Japan, Inc. (Tokyo, Japan), and were maintained in a conventional animal room equipped with an air-cleaning device.

Restraint Stress

Rats were restricted by wrapping each with a thin polycarbonate sheet and laying it over the lower electrode for 30 min.

Experimental Design

The effect of EF on restraint stress was determined as described below. To assess the restraint procedure using thin polycarbonate sheets, 6 rats were divided into two groups, restraint alone and restraint plus diazepam treatment. To examine the effect of exposure to EF, we used normal and ovariectomized rats. Normal rats were divided into two groups of restraint alone and restraint plus EF. Furthermore, ovariectomized rats were also divided into 4 sub-groups as follows: sham EF exposed (A1), sham EF exposed with restraint (A2), EF exposed with restraint (A3), sham EF exposed with diazepam treatment and restraint (A4).

Ovariectomies were performed 4 weeks before experimentation. EF exposure and restraint treatment applied in this study were as follows: Rats were exposed to 50 Hz, 17,500 V/m EF for a total of 1 hr. Rats were restrained with thin polycarbonate sheeting for the latter half of the EF exposure period. The experimental design in the control groups was the same as in the experimental group except for the absence of EF exposure.

Collecting Blood Samples

1 ml of blood was collected from subclavian vein before the initiation of experimentation and plasma prepared by centrifugation at 1,500×g for 10 minutes at 4° C. Plasma was stored at −80° C. prior to hormone measurement. After the experiment, 3 ml of whole blood from each rat was collected into a glass tube containing 9 mg EDTA by cardiac puncture under an anesthesia. 1 ml of blood was applied to analyze blood condition. Another 2 ml was centrifuged (1,500×g for 10 min. at 4° C.) and the supernatant stored at −80° C. until the measurement of hormone, glucose, lactate and pyruvate.

Blood Analyses

Hematological analyses including red and white blood cell count, platelet count, hematocrit and hemoglobin levels were performed using an automatic multi-hemocytometer (Sysmec CC-78, Sysmec inc., Tokyo, Japan). Plasma glucose, lactate and pyruvate levels were measured with an automatic analyzer (7170 Hitachi, Hitachi Co. ltd., Tokyo, Japan). ACTH levels were measured by using an ACTH radio immunoassay kit (ACTH IRMA, MITSUBISHI CHEMICAL Co. Ltd.) and a gamma counter (Auto-Gamma 5530 Gamma Counting System, Packard Instrument Co. ltd.). Plasma corticosterone level was measured using a commercial kit (ImmuChem Double Antibody Corticosterone kit, ICN Biomedicals Inc.).

Statistical Analysis

Results were expressed as mean±standard error of means (S.E.) or the data set as median, 25^{th} percentile, 75^{th} percentile, minimum and maximum values. Statistical significance of difference between paired groups was calculated by Student's t test, and the significance was defined as P<0.05. All computations for the statistical analysis were carried out in MS-EXCEL® Japanese Edition (Microsoft Office software: Ver. 9.0.1, Microsoft Japan Inc. Tokyo, Japan).

Results

Changes in Plasma ACTH Levels Induced by Restraint Stress

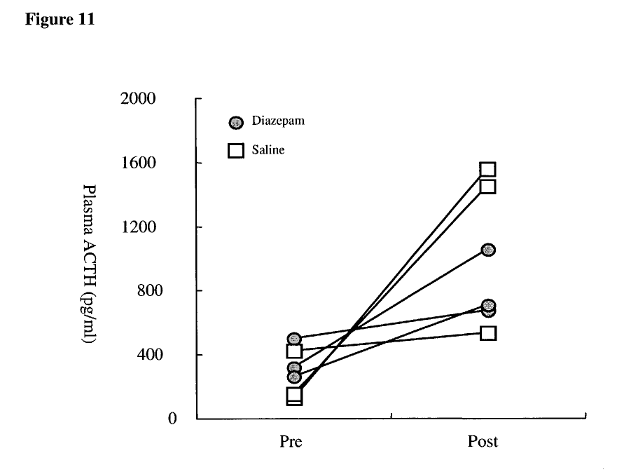

FIG. 11 displays the effect of stress on plasma ACTH levels. Rats were administrated intraperitoneally with 1 mg/kg B.W. of diazepam (filled circle) or saline (open square). Thirty minutes after diazepam administration was performed, the rats were restrained to provoke a stress response. FIG. 11 shows the ACTH level of individual rats 30 min after the start of the restraint. Pre- and Post-restraint period values (mean±S.E.) were 231±135 and 1177±325 pg/mil in the restraint alone group, and were 358±73 and 810±121 pg/ml in restraint plus diazepam group. Comparing the ACTH levels of pre- and post-restraint stress in each group, the 30 min restraint increased the plasma ACTH levels 5.1-fold and 2.3-fold higher in the restraint alone and the restraint+diazepam groups, respectively.

Effect of EF Exposure on Restraint-Induced Changes of Plasma ACTH Level

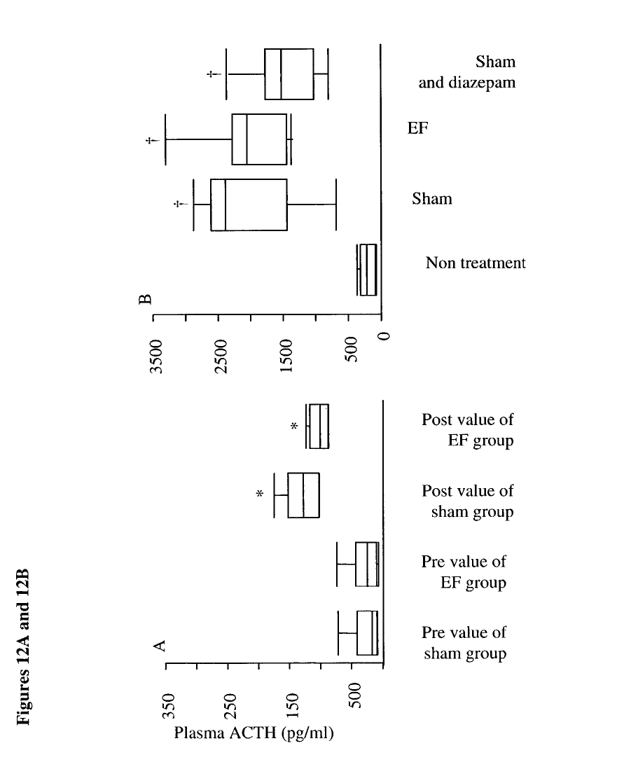

FIGS. 12A and 12B show the effect of exposure to EF on plasma ACTH level in normal (A) and ovariectomized rats (B). All rats were restrained for the latter half of the EF exposure period. Plasma ACTH levels were measured 60 min before and after EF exposure in the following groups: non-treatment (n=6), restraint alone (Sham, n=6), restraint during EF (EF, n=6) and restraint during sham EF and diazepam (Sham and diazepam, n=6). Addition of diazepam occurred 30 min before start of the EF session. Data is expressed in boxes, wherein the horizontal line that appears to divide each main box into two smaller boxes represents the median, the horizontal line that forms the bottom side of each main box represents the 25th percentile, the horizontal line that forms the top side of each main box represents the 75th percentile, the horizontal line that appears above each main box represents the maximum value, and the horizontal line that appears below each main box represents the minimum value. Pre values are not shown. *: P<0.05 from pre value. †: P<0.05 from non-treatment group.

In ovariectomized rats, plasma ACTH level in the non-restraint group did not show any changes during 60 min. In the other three groups, ACTH levels were elevated during the restraint period (FIG. 12B). Comparing among pre- and post-session, the plasma level elevated 18.6, 13.4 and 13.7-fold in the “restraint alone”, the “restraint and EF”, and the “restraint and diazepam” groups, respectively.

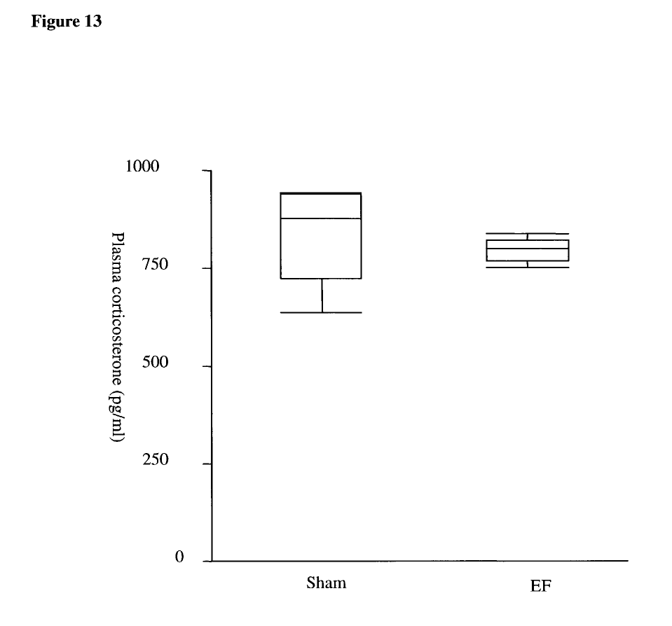

FIG. 13 shows the effect of EF exposure on plasma ACTH levels in normal rats (n=6). Data was expressed as a median, 25th percentile, 75th percentile, minimum and maximum value. FIGS. 12A and 13 show the changes in plasma level of ACTH and corticosterone in normal rats. ACTH levels in the “restraint alone” and the “restraint and EF” groups were 1595±365 and 1152±183 (pg/ml), and Corticosterone levels were 845±48 and 786±24 (ng/ml), respectively.

Effect of EF Exposure on Plasma Parameters

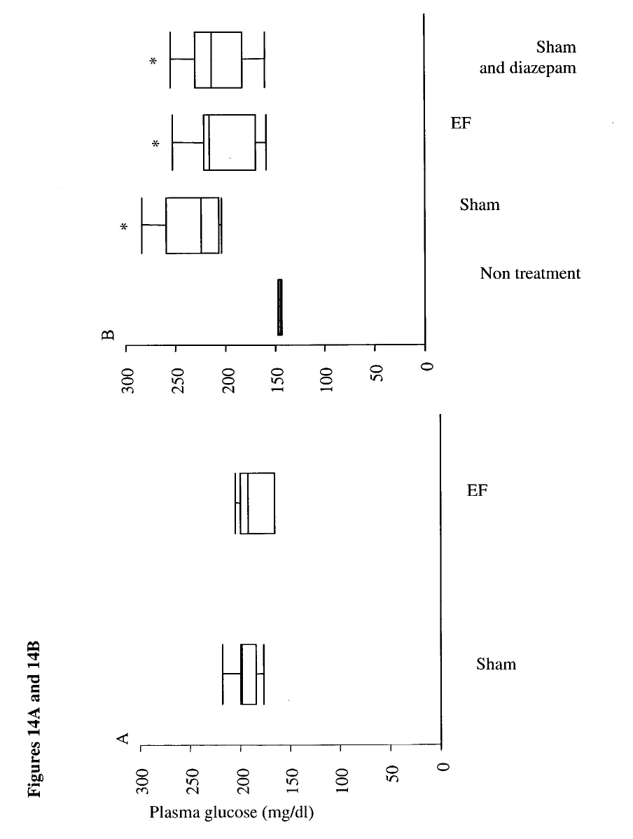

FIGS. 14A and 14B show the effect of EF exposure on restraint-induced plasma glucose level changes on normal (A) and ovariectomized rats (B). Those levels were examined after the session for 60 min (n=6). Sample number was 6 in all groups. Data was expressed as a median, 25th percentile, 75th percentile, minimum and maximum value. *: P<0.05 from non-treatment group.

In ovariectomized rats, the restraint increased the plasma glucose level (P<0.05: Student's t test), and EF or diazepam had the tendency to suppress these increases (FIG. 14B). However, the trend of suppression of plasma glucose levels in the EF group was not observed in normal rats that did not receive an ovariectomy (FIG. 14A).

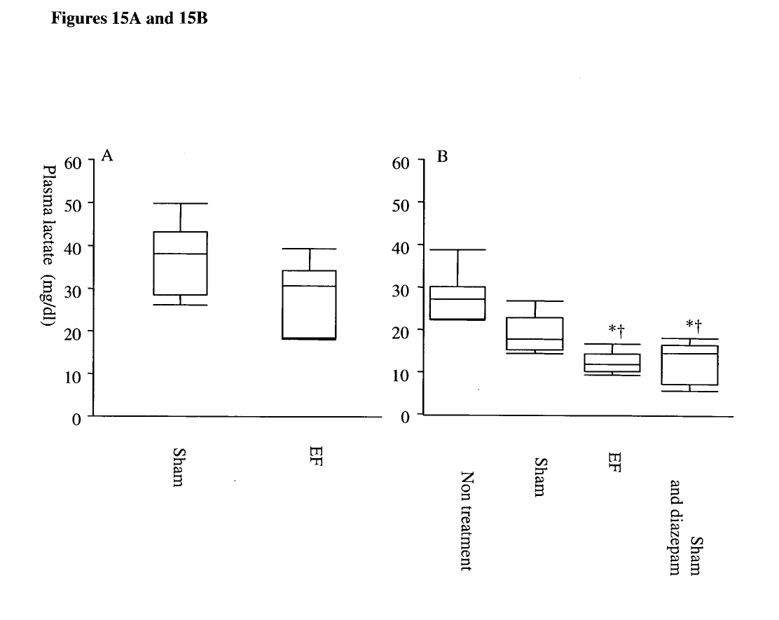

FIGS. 15A and 15B show the effect of EF exposure on restraint-induced plasma lactate levels in normal (A) and ovariectomized rats (B). The levels were measured after a 60 minute session (n=6). Data was expressed as a median, 25th percentile, 75th percentile, minimum and maximum value. *: P<0.05 from non-treatment group. †: P<0.05 from Sham group. In ovariectomized rats, plasma lactate levels in the restraint alone group did not show significant differences compared to the non-treatment group (FIG. 15B). Plasma lactate levels in the EF-exposed and the diazepam administered groups were significantly lower than those of the restraint alone group (P<0.05: Student's t test) (FIG. 15B). In normal rats, plasma lactate levels (mean±S.E.) in the presence and the absence of EF were 28.6±3.6 and 38.1±3.7 (mg/dl), (FIG. 15A). As a result of statistical analysis, lactate levels in animals exposed to EF were significantly lower than those of the restraint alone group (P<0.05: Student's t test).

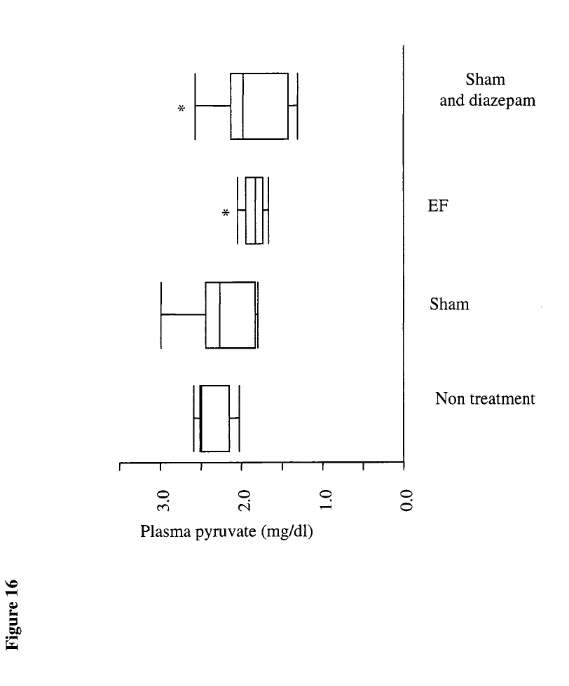

FIG. 16 shows the effect of EF exposure on restraint-induced plasma pyruvate levels in ovariectomized rats. The levels were examined after a 60 minute session (n=6). Data was expressed as a median, 25th percentile, 75th percentile, minimum and maximum value. *: P<0.05 from non-treatment group. In ovariectomized rats, plasma pyruvate levels in the restraint alone group was not significantly different from that of the non-treatment group, but tended to decrease by restraint. Subjects in groups exposed to EF or administered diazepam were significantly lower than those of sham EF exposure group (P<0.05: Student's t test) (FIG. 16).

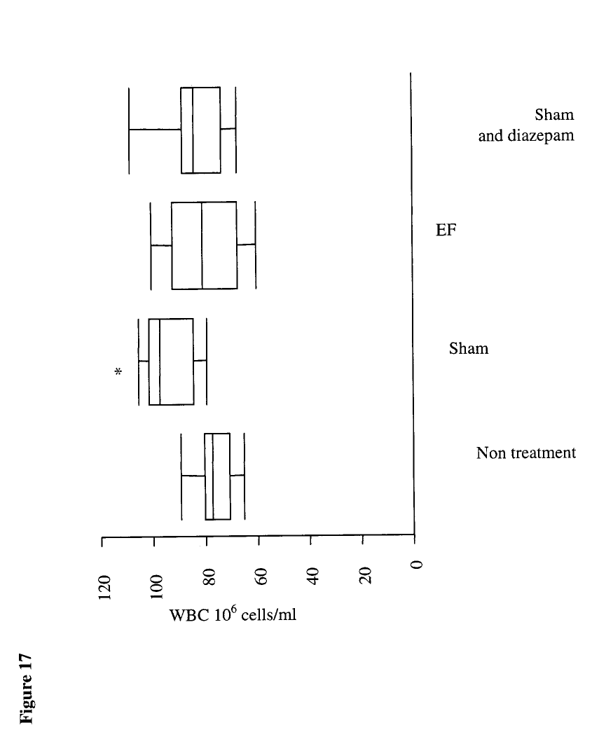

FIG. 17 shows the effect of EF exposure on restraint-induced white blood cell (WBC) counts in ovariectomized rats. The levels were examined after a 60 minute session (n=6). Data was expressed as a median, 25th percentile, 75th percentile, minimum and maximum value. *: P<0.05 from non-treatment group. Generally, the observed restraint-dependent changes related to the number of white blood cells (WBC). WBC counts in the non-treatment, restraint alone, exposure to EF, and administered diazepam groups showed 78, 99, 96 and 85 (×10² cells/μl), (FIG. 17). As a result of statistical analysis, WBC levels in animals restrained were significantly higher than those of the non-treatment group (P<0.05: Student's t test) in ovariectomized rats. WBC levels in EF exposed or diazepam administered groups tended to be higher than the non-treatment group, and were lower than the restraint alone group.

### EXAMPLE 7

Electroencephalogram Studies

Six rats were exposed to an electric field estimated at 17,500 V/m for 15 minutes a day for 7 days. The device used to expose the animals was a Healthtron Exposure Cage (described previously). Six rats were used as controls (sham-exposed). The following parameters (endpoints) were observed: brain wave abnormalities detection; percentage of each EEG level group (awake, rest, slow wave light sleep, slow wave deep sleep, and fast wave sleep); and the percentage of the frontal cortex EEG power spectrum delta (1-3.875 Hz), theta (4-15.875 Hz), alpha (8-12 Hz), beta 1 (12.125-15.875 Hz), and beta 2 (16-25 Hz). In repeated exposures at 7,000 V (17,500 V/m) for 15 minutes, a significant increase of the slow wave light sleep level was observed for a period of 1-2 hours on the first day. On day 7, significant decreases of rest stage 0-30 minutes post-exposure and awake stage were observed. A significant decrease in the awake stage and a significant increase in the slow wave light sleep stage were observed for a period ranging from 0.5-1 hour following exposure. A significant decrease in the awake stage and a significant increase of slow wave deep sleep stage were observed in period ranging from 1-2 hours following exposure. Moreover, a significant increase in the slow wave light sleep stage was observed for a period ranging from 2-4 hours following exposure.

No spontaneous EEG wave type or behavior abnormality was observed. There were no indications in this study that repeated exposure to an electric field presented any neurological concern on frequency analysis of frontal cortex in rats.

D. Additional Disorders or Conditions

For treating electrolyte imbalance, the mean induced current density generated over the cell membranes is preferably about 0.4 mA/m² to about 6.0 mA/m², more preferably about 0.4 mA/m² to about 5.6 mA/m², and still more preferably about 0.43 mA/m² to about 5.55 mA/m².

For treating arthritis, the mean induced current density generated over the cell membranes is preferably about 0.02 mA/m² to about 0.4 mA/m², more preferably about 0.025 mA/m² to about 0.35 mA/m², most preferably about 0.026 mA/m² to about 0.32 mA/m².

For treating excessive body weight, the mean induced current density generated over the cell membranes is preferably about 0.02 mA/m² to about 1.5 mA/m², more preferably about 0.02 mA/m² to about 1.2 mA/m², most preferably about 0.024 mA/m² to about 1.12 mA/m².

The invention is also useful for the prevention or treatment of musculo-skeletal and connective tissue disorders. These disorders include, for example, osteoporosis (including senile, secondary, and idiopathic juvenile), bone-thinning disorders, celiac disease, tropical sprue, bursitis, scleroderma, CREST syndrome, Charcot's joints, proper repair of fractured bone, and proper repair of torn ligaments and cartilage. The invention is also useful for rheumatoid arthritis, immunosuppression disorders, neuralgia, insomnia, headache, facial paralysis, neurosis, arthritis, joint pain, allergic rhinitis, stress, chronic pancreatitis, DiGeorge anomaly, endometriosis, urinary tract obstructions, pseudogout, thyroid disorders, parathyroid disorders, hypopituitarism, gallstones, peptic ulcers, salivary gland disorders, appetite disorders, nausea, vomiting, thirst, excessive urine production, vertigo, benign paroxysmal positional vertigo, achalasia and other neural disorders, acute kidney failure, chronic kidney failure, diffuse esophageal spasms, and transient ischemic attacks (TIAs). The invention is also useful for the treatment of additional renal disorders involving osmolality, maintenance thereof and conditions or disorders involving an osmolar imbalance.

E. EF Therapy Apparatus

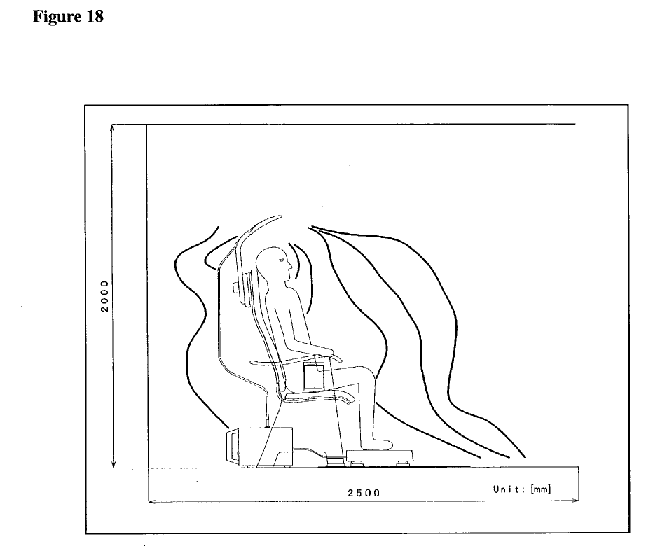

EF apparatuses are designed to generate an electric field in which the individual is placed. As demonstrated by FIG. 18, the electric field may encompass the entire subject. Alternatively, the field may encompass only a particular region or organ of the subject.

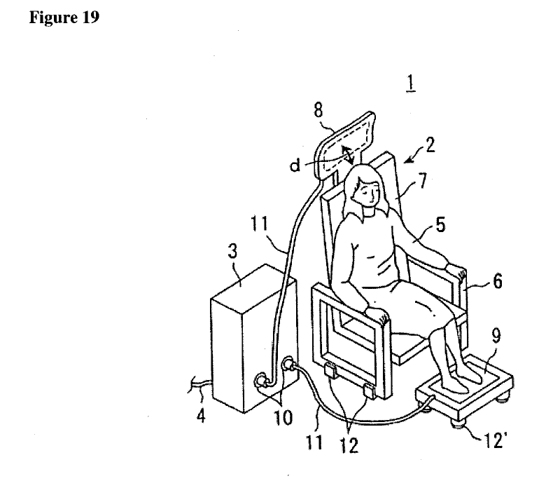

FIG. 19 is a schematic view of a high voltage generation apparatus (1) showing an embodiment of the present invention. Namely, the electric potential therapy apparatus (1) comprises an electric potential treatment device (2), a high voltage generation apparatus (3) and a commercial power source (4). The electric potential treatment device (2) comprises a chair (7) with armrests (6) where a subject (5) sits, a head electrode (8) as an opposed electrode attached to the upper end of the chair and arranged above the top of the subject's head (5), and a second electrode (9) as ottoman electrode which is a main electrode where the subject (5) puts his/her legs on the top face thereof. Note that the head electrode (8), as an opposed electrode of the second electrode (9), which is a main electrode, may otherwise be ceiling, wall, floor, furniture or other contents or parts of the room. The high voltage generation apparatus (3) generates a high voltage to impress a voltage to the head electrode (8) and second electrode (9). The high voltage generation apparatus (3) is generally installed under the chair (7), between the legs and on the floor, or in the vicinity of the chair (7). A distance (d) between the first or head electrode (8) and the top of the patient's head can be varied. An insulation material surrounds the head electrode (8) and the second electrode (9). This second electrode (9) is connected to a high voltage output terminal (10) of the high voltage generation apparatus (3) by an electric cord (11). It is also provided with the high voltage output terminal (10) to impress a voltage to the head electrode (8) and the second electrode (9). In addition, the chair (7) and the second electrode (9) comprise insulators (12), (12)′ at the contact positions with the floor. The distance (d) between the human body surface and the first electrode (8a) can be changed easily by putting cushions of different thickness on the bed base (31).

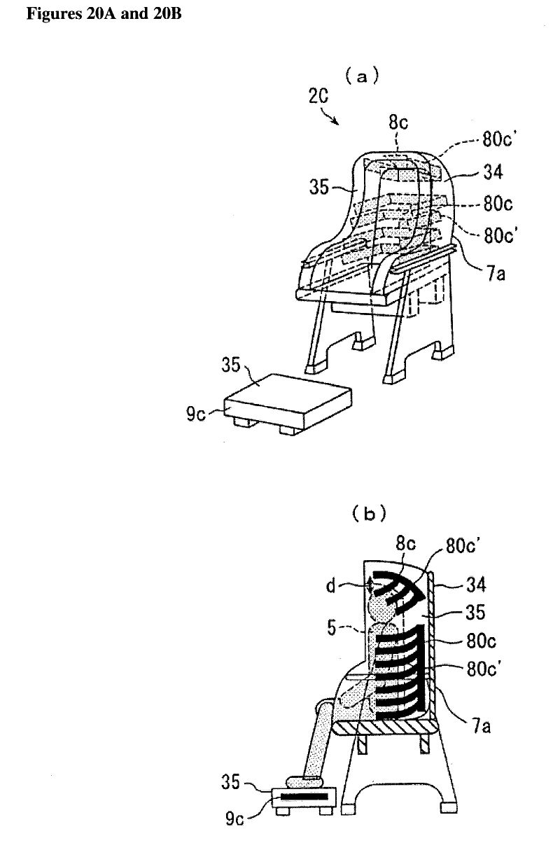

An electric potential treatment device (2C) provided with still another structure has a chair type shown in FIG. 20A [perspective view] and FIG. 20B [side view illustrating the positional relationship between the subject (5) and respective electrodes painted in black]. The chair (7a) is provided with a front open cover body (34) covering the subject (5). This cover body (34) is provided with a first electrode (8c) as an opposed electrode to receive the head of the subject (5), a second electrode (9c) which is an ottoman electrode as main electrode, and another first electrode (80c) disposed at the position of shoulder to waist of the sitting posture as an opposed electrode disposed at the waist upper body portion. The other first electrode (80c) has a plurality of side electrodes (80c′) so as to cover the body of the subject (5) from the side. Preferably, the first electrode (8c) is arranged along the human body head portion, and another first electrode (80c) is disposed in a plurality of stages along the longitudinal direction from both shoulders to the waist. These first electrode (8c), another first electrode (80c), the side electrodes (80c′) and second electrode (9c) are arranged in an insulating material (35). A detachable cushion member made of insulator is attached to the cover body (34). Thus, the attachment of a cushion member, available in different degrees of thickness, can vary the distance between the human body surface and the first electrodes (8c), (80c), (80c′). In such electric potential treatment device (2c) also, as mentioned above, the induced current control means can control the body surface electric field and flow an extremely small amount of induced current in the respective areas of a human body trunk by making the applied voltage to be applied to the first electrodes (8c), (80c), (80c′) as an opposed electrode, and the second electrode (9c), and the distance (d) between the first electrode (8c), (80c), (80c′) and the human body trunk surface variable, or by controlling the applied voltage to be applied to the first electrode (8c), (80c), (80c′) and second electrode (9c) and further, by changing the distance (d) between the first electrode (8c), (80c), (80c′) and the human body surface.

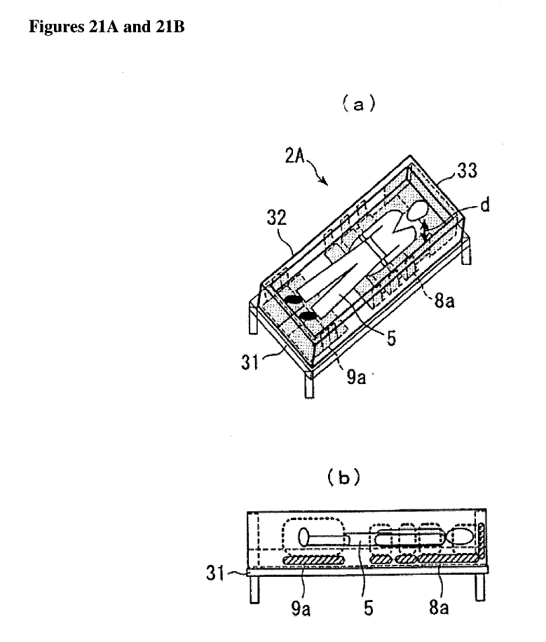

An electric potential treatment device (2A) provided with another structure is shown in FIG. 21A [perspective view] and FIG. 21B [side view]. This electric potential treatment device (2A) has a bed type. A box (32) for containing the subject (5) is disposed on a bed base (31). Respective electrodes are provided in this box (32). In short, it is provided with a first electrode (8a) as an opposed electrode and a second electrode (9a) placed at a leg portion of the human body as main electrode. The first electrode (8a) is placed at head, shoulders, abdomen, legs and hips of a human body or other areas. And preferably, the first electrode (8a) has the shape, breadth and area approximately equal to head, shoulders, abdomen and hips of a human body. Blank areas in these drawings show the points where no electrodes are disposed. Electrodes are disposed in an insulator (33). A cushion made of an insulator (not shown) is put on the respective electrodes on the bed base (31). There, cushions of different thickness are prepared.

In FIG. 19 mentioned above, the distance (d) between the head electrode (8) above the head and the human body trunk surface of the subject (5) is set to about 1 to 25 cm, in FIG. 20A, the distance (d) between the first electrode (8c), (80c), (80c′) and the subject (5) human body trunk surface is set to about 1 to 25 cm, preferably about 4 to 25 cm, and in FIG. 21A, the distance (d) between the first electrode (8a), (8b) and the human body trunk surface of the subject (5) to about 1 to 25 cm, preferably about 3 to 25 cm.

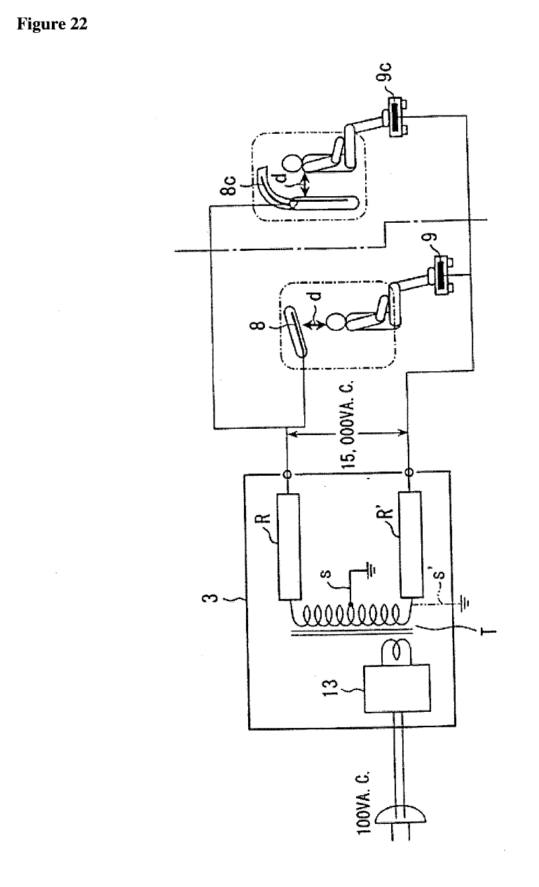

The high voltage generation apparatus (3) has, as described below for an electric configuration block diagram in FIG. 22, a booster transformer (t) for boosting a voltage of the commercial power source 100V AC to, for example, 15,000 V, and current limitation resistors (R), (R)′ for controlling the current flowing to the respective electrodes. This high voltage generation apparatus (3) has a configuration wherein a middle point (s) of a booster coil (T) is grounded, and the ground voltage is set to half of the boosted voltage. As shown by the illustrated provisory line, a point (s′) can be grounded. Here, as the block diagram shown in FIG. 22, a high voltage whose high voltage side middle point (s) is grounded by the booster transformer (T) is obtained from an 100V AC power source passing through a voltage controller (13) of the high voltage generation apparatus (3) and further, respective high voltages are connected to the head electrodes (8), (8c) or the like (see below) and the second electrodes (9), (9c) or the like (see below) through the current limitation resistors (R), (R′) for human body protection. And, the electric potential therapy apparatus (1) is provided with induced current control means. This induced current control means can cause an extremely small amount of induced current to flow in respective areas composing a human body trunk of the subject (5) with control of the body trunk electric field by varying the applied voltage to be applied to the head electrode (8) and second electrode (9), and a distance (d) between the head electrode (8) and the human body trunk surface, or by controlling the applied voltage to be applied to the head electrode (8) and second electrode (9), or further by varying the distance (d) between the head electrode (8) and the human body trunk surface. The distance (d) between the human body surface and the first electrode (8a) can be changed easily by putting cushions of thus different thickness on the bed base (31).

By increasing the induced current even in a state where a high voltage is applied in the electric potential therapy apparatus (1), a higher therapeutic effect can be obtained, even for the same period of time equal to that in the conventional method. In addition, the treatment can be completed within a time shorter than before. And further, to obtain the same therapeutic effect, an induced current of the same value as the prior art can be obtained with a lower voltage and in a same treatment time as before.

The electric potential therapy apparatus (1) of the present invention is designed to be exempt, as much as possible, from high output electronic noise, high-level radio frequency noise and strong magnetic field. In order to reduce the influence of electromagnetic field interference with the electric potential therapy apparatus (1), it is preferable to use driven mechanical switch, relay and electric motor or electric timer or other electric components rather than electronic components, semiconductor, power component (such as thyristor, triac) electronic timer or EMI sensible microcomputer for the designing and manufacturing thereof. However, as electronic functional component, the electronic serial bus switching regulator for optical emitter diode power source is effective, and this optical emitter diode is used as an optical source for informing the subject or the operator of the active or inactive state of the electric potential therapy apparatus of the present invention.

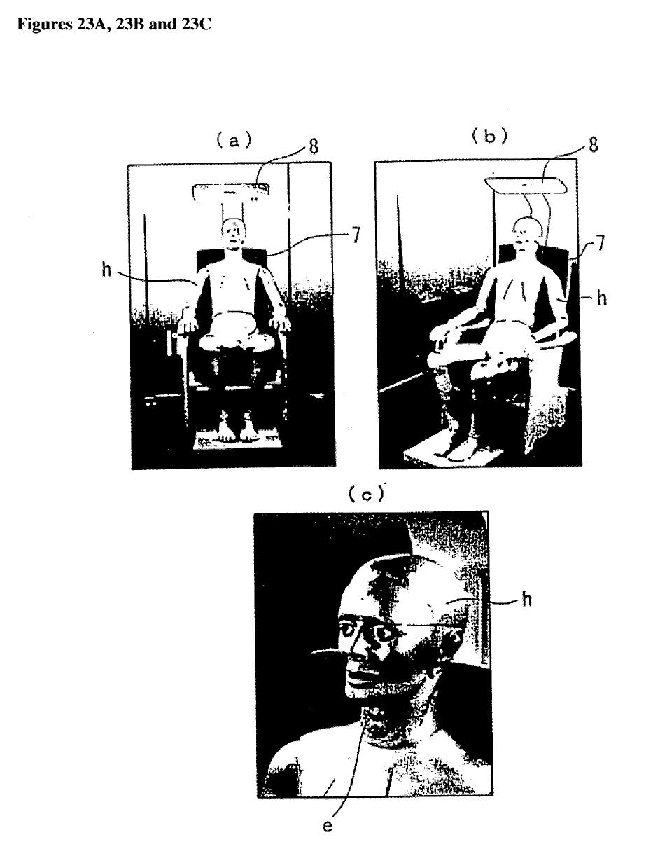

As mentioned above, a simulated human body (h) can be used to measure the EF and induced current, as shown in FIGS. 23A, 23B and 23C. This simulated human body (h) is made of PVC and the surface thereof is coated with a mixed solution of silver and silver chloride. This makes the resistance (1K Ω or less) equivalent to the resistance of a real human body. Simulated human body (h) is used worldwide as a nursing simulator, and its dimensions resemble those of an average human body, for example, it is 174 cm tall. The dimensions are further described in Table 1.

TABLE 1
Measurement of Current Density in Simulated Human Body

| | Circumference | Cross Sectional Area |
|---|---|---|
| Section of Area | (mm) | (m²) |
| Eye | 550 | 0.02407 |
| Nose | 475 | 0.01795 |
| Neck | 328 | 0.00856 |
| Chest | 770 | 0.04718 |
| Pit of the stomach | 710 | 0.04012 |
| Arm | 242 | 0.00466 |
| Wrist | 170 | 0.00230 |
| Trunk | 660 | 0.03466 |
| Thigh | 450 | 0.01611 |
| Knee | 309 | 0.00760 |
| Ankle | 205 | 0.00334 |

The body surface electric field is measured by attaching a disk shaped electric field measurement sensor (e) to a measurement area of the simulated human body (h). The measurements occur under the condition of 115 V/60 Hz and 120 V/60 Hz.

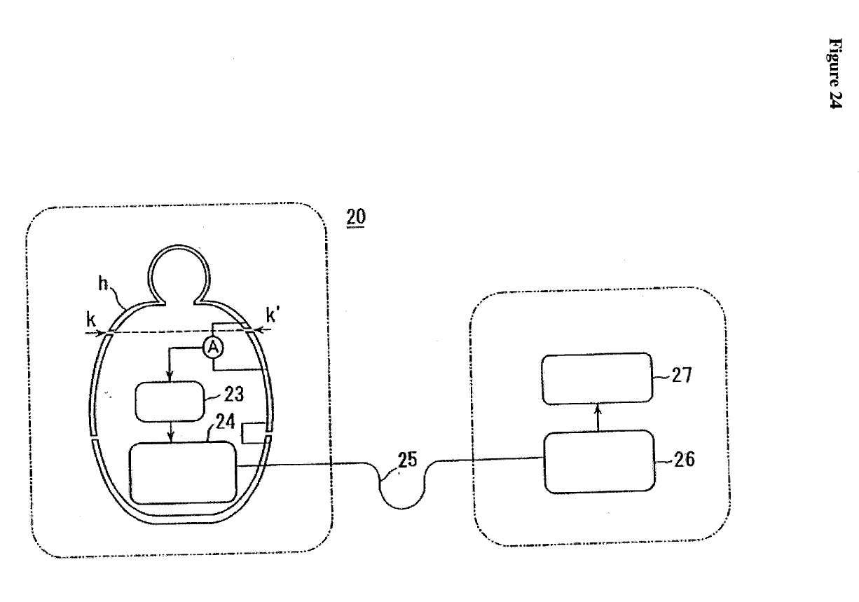

A method of measuring an induced current, and an apparatus therefor, are shown in FIG. 24. In the induced current measurement apparatus (20), as shown in FIGS. 23A and 23B, the simulated human body (h) is put on the chair (7) in a normal sitting state. The head electrode (8) over the head, which is the opposed electrode, is adjusted and installed to be 11 cm from above a head of the simulated human body (h). The measurements are achieved by measuring respective portions such as, for example, the illustrated k-k′ line portion in FIG. 24, transferring the induced current waveform through optical transfer, and observing this waveform at the ground side of the induced current measurement apparatus (20). Here, the applied voltage is 15,000 V. In this measuring method, the measurement of the current induced at the section of respective areas of the simulated human body (h) obtains the induced current by creating a short-circuit (22) [not shown] of a current flowing across the section of the simulated human body (h) using two lead wires. The measured induction current is converted into a voltage signal through an I/V converter (23) (FIG. 24). Next, this voltage signal is converted into an optical signal by an optical analog data link at the transmission side.

These optical signals are transferred to an optical analog data link (26) at the reception side, through an optical fiber cable (25) and converted into a voltage signal. This voltage signal is then processed by a frequency analyzer (27) for frequency analysis by a waveform observation and analysis recorder. A buffer and an adder are disposed between the I/V converter (23) and the optical analog data link (24) at the transmission side [not shown]. Thus, electric field value and induction current measured at the 115 V/60 Hz and 120 V/60 Hz, at the position of respective areas of the simulated human body (h), are shown in Table 2. If the electric field value is different from this Table 2, accordingly, it is known that the induced current value flowing there is also different. Therefore, it is supposed that it is evident that the induced current effective for respective areas of a real human body trunk can be obtained by changing the electric field of the concerned respective areas.

TABLE 2
Relationship between Electric Field Value and Induced Current Value

| | @ 115 V/50 Hz | | @ 120 V/60 Hz | |
|---|---|---|---|---|
| | Electric Field | Induced | Electric Field | Induced |
| Section of | Value | Current | Value | Current |
| Area | (kV/m) | (μA) | (kV/m) | (μA) |
| Top of the | 182 | 0.72 | 90 | 0.90 |
| head | | | | |
| Front of the | 81 | 0.32 | 84 | 0.40 |
| head | | | | |
| Back of the | 113 | 0.44 | 118 | 0.55 |
| head | | | | |
| Side of the | 16 | 0.06 | 16 | 0.08 |
| neck | | | | |
| Shoulder | 37 | 0.15 | 38 | 0.18 |
| Chest | 19 | 0.08 | 20 | 0.10 |
| Arm | 29 | 0.11 | 30 | 0.14 |
| Elbow | 33 | 0.14 | 34 | 0.17 |
| Back | 52 | 0.20 | 54 | 0.25 |
| Back of the | 21 | 0.08 | 22 | 0.10 |
| hand | | | | |
| Coccyx | 42 | 0.17 | 43 | 0.21 |
| Knee | 11 | 0.05 | 12 | 0.06 |
| Patella | 21 | 0.08 | 22 | 0.10 |
| Tip of the | 3.4 | 0.01 | 3.5 | 0.02 |
| foot | | | | |
| Bottom of the | 348 | 1.37 | 363 | 1.72 |
| foot | | | | |

The body surface electric field E can be obtained by using the following equation, from the induced current value of the respective areas obtained by the measurement method of the induced current of respective areas shown in FIG. 24. Namely, E=I/εoωS. Here, S is a section of the electric field measurement sensor, εo is an induction rate in a vacuum, I is an induced current, ω is 2πf and f is frequency. When the induced current of respective areas is obtained by the aforementioned method, an induced current density J of respective areas can be obtained using the following expressions. Namely, A=2πr, B=πr², B=A²/4π, J=I/B, where A is a circumference, B is a circle area, r is a radius, I is a measured current, and J is an induced current density.

The induced current control means mentioned above can cause an extremely small amount of induced current to flow in respective areas of a human body trunk, when the electric potential therapy is performed, by controlling the voltage of the head electrode (8) and the applied voltage applied to the second electrode (9).

Table 3 shows the relationship among: (1) the induced current (μA) at the nose, neck and trunk, (2) the induced current density (mA/m²) at the nose, neck and trunk, and the applied voltage (KV) at 120V/60 Hz. Under the same applied voltage, the current density tends to be highest in the neck, next highest in the trunk and lowest in the nose. Note that the induced current densities in Table 3 are less than 10 mA/m² and that current densities of 10 mA/m² or less have been established as safe by the International Commission on Non Ionizing Radiation Protection.

TABLE 3
Applied Voltage and Induced Current

| | Induced current Value (μA) | | | Induced Current Density (mA/m²) | | |
|---|---|---|---|---|---|---|
| Applied | Head | | | Head | | |
| voltage | Portion | Neck | Trunk | Portion | Neck | Trunk |
| [kV] | (nose) | Portion | Portion | (nose) | Portion | Portion |
| 0 | 0 | 0 | 0 | 0.0 | 0.0 | 0.0 |
| 5 | 10 | 11 | 30 | 0.6 | 1.3 | 0.9 |
| 10 | 20 | 23 | 61 | 1.1 | 2.6 | 1.7 |
| 15 | 30 | 34 | 91 | 1.7 | 3.9 | 2.6 |
| 20 | 40 | 45 | 121 | 2.2 | 5.2 | 3.5 |
| 25 | 50 | 57 | 152 | 2.8 | 6.6 | 4.4 |
| 30 | 60 | 68 | 182 | 3.3 | 7.9 | 5.2 |

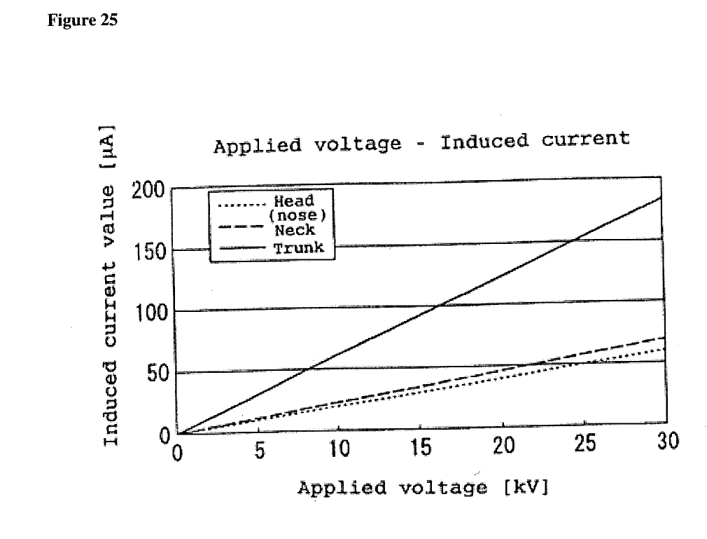

FIG. 25 also shows the relationship between the applied voltage (KV) and the induced current (μA) in the nose, neck and trunk. As evident in FIG. 25, the applied voltage and the induced current are proportional to each other.

Table 4 shows the variation of induced current and induced current density in the neck of a human as a function of the distance (d) between the head electrode (8) and the top of the head.

TABLE 4
Change in Induced Current as Function of Distance from Electrode

| Distance of First Electrode | | |
|---|---|---|
| from Top of Head | | Induced |
| Distance | Induced Current Value | Current Density |
| (cm) | (μA) | (mA/m²) |
| 4.3 | 50 | 5.8 |
| 5.4 | 46 | 5.4 |
| 6.3 | 43 | 5.0 |
| 6.9 | 40 | 4.7 |
| 8.3 | 39 | 4.5 |
| 9 | 38 | 4.4 |
| 9.9 | 35 | 4.1 |
| 11 | 34 | 3.9 |
| 12 | 34 | 3.9 |
| 13 | 33 | 3.8 |
| 14 | 31 | 3.7 |
| 15 | 30 | 3.5 |
| 16.1 | 30 | 3.5 |
| 17.2 | 30 | 3.5 |

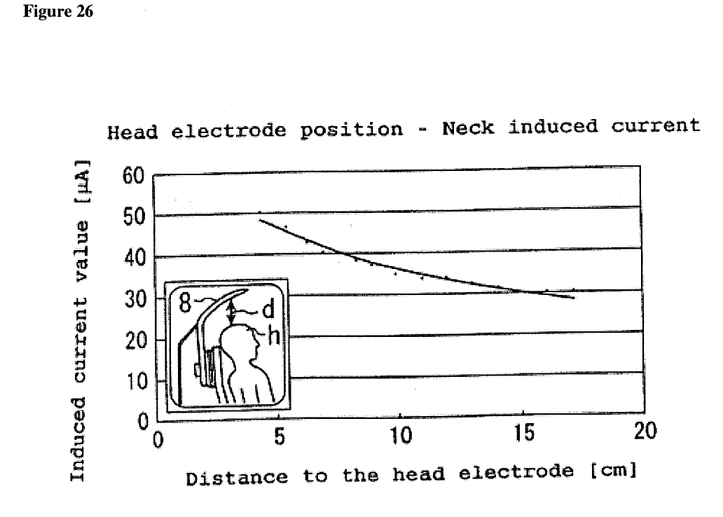

Table 4 indicates that, at a distance of 15 cm or more, the induced current stabilizes at 30 μA. Thus, to vary the induced current by varying distance, the distance should be 15 cm or less. FIG. 26 also shows the variation of induced current depending on the distance (d).

In an experiment involving about 300 cases of lumbago in humans, we determined that EF was effective in treating lumbago. We also determined the optimal dosage and parameters as follows. In short, the optimal dose amount is obtained by controlling the product of the induced current value flowing in areas composing a human body trunk and the induced current flowing time. Otherwise, it is obtained by controlling the product of the applied voltage sum of the first electrode voltage and the second electrode voltage, and the applying time thereof. For lumbago, the therapeutic effect of EF is optimized by applying it for about 30 min at a voltage of about 10 KV to about 30 KV, preferably about 15 KV. In other words, at about 300 KV/min to about 900 KV/min, preferably about 450 KV/min.

Here, Table 5 shows the induced current value measured with 115 V/50 Hz at the section of respective areas composing the trunk of the simulated human body (h), and the induced current density obtained by calculation from this induced current value, taking the dimensions of the simulated human body (h) of the Table 1 into consideration. From Table 5, measured values of induced current (μA) in respective areas composing the trunk of human body and the calculated values of induced current density (mA/m²) are as follows: eye; 18/0.8, nose; 24/1.3, neck; 27/3.1, chest; 44/0.9, pit of the stomach; 8.6/1.6, and trunk; 91/2.8.

TABLE 5
Area, Induced Current Value, and Induced Current Density

| | Induced Current | Induced Current Density |
|---|---|---|
| | @ 115 V/50 Hz | @ 115 V/50 Hz |
| Section of Area | (μA) | (mA/m²) |
| Eye | 18 | 0.8 |
| Nose | 24 | 1.3 |
| Neck | 27 | 3.1 |
| Chest | 44 | 0.9 |
| Pit of the stomach | 65 | 1.6 |
| Arm | 8.6 | 1.8 |
| Wrist | 3.1 | 1.3 |
| Trunk | 73 | 2.1 |
| Thigh | 46 | 2.8 |
| Knee | 52 | 6.8 |
| Ankle | 58 | 17 |

Moreover, based on the aforementioned induced current and induced current density, the induced current and induced current density at 120 V/60 Hz are calculated according to the following expression 1 and expression 2.

Expression 1:

Induced Current;

I(60 Hz)=I(50 Hz)×60/50×120/115

Expression 2:

Induced Current Density;

J(60 Hz)=J(50 Hz)×60/50×120/115

Table 6 shows the calculation result of the induced current and induced current density of respective areas that are human body trunk at 120 V/60 Hz. From Table 6, measured values of induced current (μA) in respective areas composing the trunk of human body and the calculated value of induced current density (mA/m²) are as follows: Eye; 23/0.9, nose; 30/1.7, neck; 34/3.9, chest; 55/1.2, pit of the stomach; 11/2.3, and trunk; 114/3.6.

TABLE 6
Area, Induced Current Value, and Induced Current Density

| | Induced Current | Induced Current Density |
|---|---|---|
| | @ 120 V/60 Hz | @ 120 V/60 Hz |
| Section of Area | (μA) | (mA/m²) |
| Eye | 23 | 0.9 |
| Nose | 30 | 1.7 |
| Neck | 34 | 3.9 |
| Chest | 55 | 1.2 |
| Pit of the stomach | 81 | 2.0 |
| Arm | 11 | 2.3 |
| Wrist | 3.9 | 1.7 |
| Trunk | 91 | 2.6 |
| Thigh | 57 | 3.6 |
| Knee | 64 | 8.5 |
| Ankle | 72 | 22 |

When the distance between the electrode and the human body area is fixed, the above-mentioned applied voltage and the induced current flowing in the body trunk respective areas of a human body are in proportional relationship. Therefore, when a human body is treated with a chair, the optimal dose amount can be obtained by controlling the product of the applied voltage and the applying time, because the electric field intensity of respective areas of a human body is almost decided by the applied voltage, if the distance between the electrode and the human body is decided in a manner of the greatest common divisor.

A trained individual would understand that the amount of voltage applied, as well as the current density, may be controlled using an appropriate electric field apparatus, such as, a Healthtron HES-30™ Device (Hakuju Co.). For example, the induced current generated in the presence of a biological sample may be increased by raising the potential of the electrode through which the EF is applied. Other appropriate apparatuses are known to trained individuals, and include but are not limited to, the 00298 device (Hakuju Co.), the HEF-K 9000 device (Hakuju Co.), the HES-15A device (Hakuju Co.), the HES-30 device (Hakuju Co.), the AC/DC generator (Sankyo, Inc.), and the Function generator SG 4101 (Iwatsu, Inc.). Some features of exemplary apparatuses are presented in Table 7 along with the specifications for those apparatuses.

Additional electric field apparatuses useful with the methods of the invention include the electric field generating apparatus disclosed in U.S. Pat. No. 4,094,322, herein incorporated by reference in its entirety. This therapeutic apparatus enables the directed delivery of an electric field to a desired part of a patient lying on the apparatus. Other electric field apparatus are disclosed in U.S. Pat. No. 4,033,356, U.S. Pat. No. 4,292,980, U.S. Pat. No. 4,802,470, and British Patent GB 2 274 593, each of which is herein incorporated by reference in its entirety.

Table 7 provides the particular specifications of selected EF apparatuses that may be used with the methods of the invention.

TABLE 7
Preferred features of EF therapy devices of the invention

| | Rated | Rated | | | | | | | | | | |
|---|---|---|---|---|---|---|---|---|---|---|---|---|
| | Power | Power | Power | | | Automatic | | | | | | |
| Type of | Supply | Supply | Con- | | | Timer | | | | | | |
| Device | Voltage | Frequency | sumption | Output Voltage | | Duration | Weight | | | | | |
| 00298 | 115 V | 60 Hz | 18 VA +/− | Upper | Charging | 30 min. | Control | Upper | Charging | Treatment | Insulating | High |
| | AC | | 15% | Electrode | Footrest | +/− 10% | Switch | Electrode | Footrest | Chair with | Mat | Voltage |
| | | | | 7500 V | 7500 V | | Box | 2 kg | 8 kg | Power off | 2 kg | Unit |
| | | | | +/− 10%, | +/− 10%, | | 3 kg | | | Switch Box | | 40 kg |
| | | | | 60 Hz AC | 60 Hz AC | | | | | 15 kg | | |

| | | | | | | | | |
|---|---|---|---|---|---|---|---|---|
| HEF-K | 100 V | 50 or 60 | 10 W | Upper | Charging | 30 min., | Chair | Main Body |
| 9000 | AC | Hz | | Electrode | Footrest | and 1, 2, | 15.8 kg | 41 kg |
| | | | | 0-3,500 V | 0-3,500 V | 4, 6, and 8 | | |
| | | | | | | hr. | | |

| | | | | | | |
|---|---|---|---|---|---|---|
| HES-15A | 100 V | 50 or 60 | 100 VA | 0-15,000 V | unlimited | 130 kg |
| | AC | Hz | | | | |
| HES-30 | 100 V | 50 or 60 | 200 VA | 0-30,000 V | unlimited | 240 kg |
| | AC | Hz | | | | |
| AC/DC | 100 V | 50 or 60 | 25 W | AC: 0-3,500 V; | | |
| Generator | AC | Hz | | DC: 0-3,500 V | | |
| Function | 100 V | 50 or 60 | 25 W | AC: 0-3,500 V; | | |
| Generator: | AC | Hz | | DC: 0-3,500 V | | |
| SG 4101 | | | | | | |

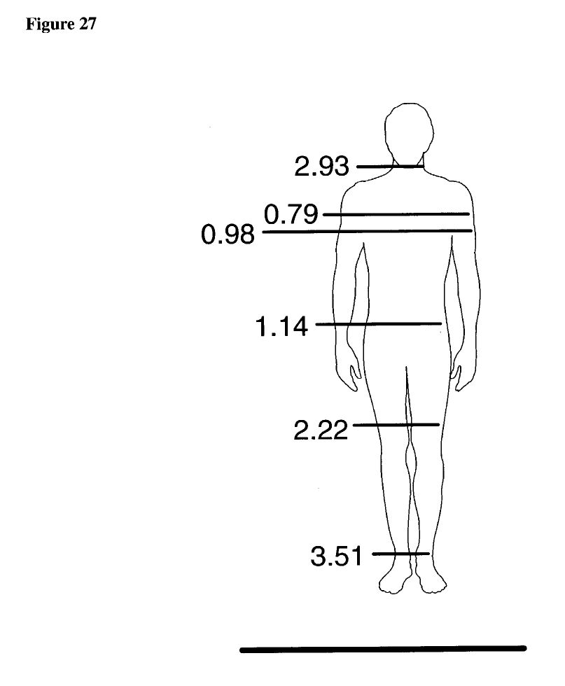

The current-density distribution induced by 60-Hz electric fields in homogeneous but irregularly shaped human models was calculated using a two-stage finite-difference procedure (Hart, F. X., Bioelectromagnetics 11:213-228 (1990)). For the case of the ungrounded human model exposed to an electric field of 10 kV/m, the induced current density in the plane through the torso at the level of the lower back was 1.14 mA/m² (FIG. 27). The current densities at other locations ranged from 0.8-3.5 mA/m². The exact values depended upon the capacitive coupling between the model and ground, but a reasonable range of coupling conditions resulted in changes of less than a factor of 2 in the calculated current densities. Similar results were found by others (Gandhi, O. P. & Chen, J. Y., Bioelectromagnetics Suppl. 1:43-60 (1992); King, R. W. P., IEEE Trans. Biomed. Eng. 45:520-530 (1998)).

The finite-difference time-domain method was used to calculate induced currents in anatomically based models of the human body (Furse, C. M. & Gandhi, O. P., Bioelectromagnetics 19:293-299 (1998)). The calculation was performed on a supercomputer, allowing much greater resolution than previously possible. The results obtained for current densities induced in specific tissues in the model are shown in Table 8. Comparable results were found by others using composite models of tissues including fat-muscle (Chuang, H.-R. & Chen, K.-M., IEEE Trans. Biomed. Eng. 36:628-634 (1989)) and bone-brain (Hart, F. X. & Marino, A. A., Med. Biol. Eng. Comp. 24:105-108 (1986)).

TABLE 8
Current densities induced in specific tissues of
human subject exposed to 60 Hz electric field of 10 kV/m.

| | Induced Current |
|---|---|
| | Density |
| Tissue | (mA/m²) |
| Intestine | 1.3 |
| Spleen | 1.4 |
| Pancreas | 1.5 |
| Liver | 1.4 |
| Kidney | 2.8 |
| Lung | 0.6 |
| Bladder | 1.9 |
| Heart | 2.2 |
| Stomach | 1.2 |
| Testicles | 0.7 |
| Prostate | 1.0 |
| Eye humor | 5.6 |
| Cerebrospinal fluid | 4.8 |
| Pineal gland | 1.4 |
| Pituitary gland | 3.5 |
| Brain | 1.9 |

### EXAMPLE 8

Exposure to Electric Field (EF): Its Palliative Effect on Some Clinical Symptoms in Human Patients

The electric field exposure apparatus, Healthtron (Model HES 30, Hakuju Institute for Health Sciences Co., Ltd., Tokyo, Japan) was used. Healthtron comprises a step-up transformer (a device for controlling the voltage in the circuit), a seat, and electrodes. It applies high voltage to one of two opposing electrodes to make a constant potential difference and form an EF in the space between the two electrodes.

The users were comfortably seated and allowed to read a book or sleep during the duration of exposure. To prevent accidental electric shocks due to formation of electric currents, the subjects were not allowed any form of bodily contact with the floor, as well as with anyone (operators and other persons exposed to electricity) during treatment. The insulator-covered electrodes were placed on the floor on which the feet were allowed to rest, and on the head of each patient. The initial power supply of 30,000-volts (ELF of 50 or 60 Hz) was applied to the electrode placed on the foot, generating an EF between the foot- and head-positioned electrodes. Exposure to electricity lasted for 30 minutes per session, and the frequency of exposure varied from once daily to once per week.

The efficacy of Healthtron was assessed based on the results obtained from questionnaires administered from Aug. 1, 1994 to Jun. 30, 1997, at the Toranomon Clinic Minato-ku, Tokyo, Japan, under the direct supervision of Yuichi Ishikawa, MD. A total of 1,253 patients (489 males; 764 females) were administered the instrument, of which 505 (208 males, 297 females), visited the clinic and used the Healthtron device and accomplished the instrument at least twice. Others may have used the device more than twice. To reduce the extent of subjectivity of the entries in the questionnaire, the evaluation of the palliative effect of Healthtron was limited to these 505 patients.

Every Healthtron user was attended to by a physician, and interviewed on the palliative effect of the instrument during the previous visit. The interview included questions on major bodily complaints (=symptoms), past medical history and treatment, frequency of utilization of Healthtron and impressions after use, including its palliative effect, and the user's personal possession of Healthtron. The severity of symptoms at the first hospital visit was rated a 3, and the severity after Healthtron therapy was classified into 5 grades, namely: very good (5); good (4); unchanged (3); aggravated (2); and highly aggravated (1). Very good and good were classified as “palliated”, and the duration of palliation in days regardless of the frequency/interval of exposure, was likewise recorded.

Results

The patients' ages ranged between 20 and 90 years old, with 85.3% comprising the >40 years age bracket (Table 9). There were 208 (41%) males and 297 (59%) females. Fifty-five different symptoms were identified, and the proportion of those patients that reported palliation per symptom with Healthtron therapy is summarized in Table 9. Symptoms that were identified by at least 10 patients included cold feeling in the extremities, fatigue, headache, hypertension, insomnia, joint pain, lower back pain, pain in the extremities, pruritus cutaneous, sensation of numbness in the extremities, shoulder/neck pain, and stiffness. The palliative effect of Healthtron therapy was evident with headache without accompanying fever, organotherapy such as subarachnoidal or cerebral hemorrhage, or inflammation (91.7%), joint pain (66.7%), low back pain (57.3%), shoulder/neck pain and stiffness (56.0-57.8%), and in alleviating fatigue (55.0%). Interestingly, the palliative effect on pain-related symptoms affecting locomotorial organs (head, joints, shoulder, neck, extremities and abdomen) was recorded in 175 (58.5%) of 299 cases. These pain-related symptoms were not ascribable to traumas. Of the 10 patients with pruritus cutaneous, while 4 claimed to have been palliated, the clinical manifestations were aggravated in one patient after the first therapy.

TABLE 9
Age range and sex distribution of Healthtron users

| Age Range | Number of Users | Male:Female |
|---|---|---|
| ˜20 | 2 | 2:0 |
| 21˜30 | 38 | 15:23 |
| 31˜40 | 34 | 10:24 |
| 41˜50 | 81 | 29:52 |
| 51˜60 | 147 | 59:88 |
| 61˜70 | 143 | 69:74 |
| 71˜80 | 50 | 20:30 |
| 81˜90 | 10 | 4:6 |
| Total | 505 | 208 (41%):297 (59%) |

Table 10 shows the palliation rate for 55 identified clinical symptoms in 505 patients.

TABLE 10
Palliation rate for 55 clinical symptoms in 505 patients

| | | No. of patients with |
|---|---|---|
| Symptoms | No. of patients | palliation (%) |
| abdominal fullness | 1 | 0 (0) |
| abdominal pain | 2 | 1 (50) |
| allergic constitution | 7 | 3 (42.9) |
| alopecia | 3 | 3 (100) |
| arrhythmia | 2 | 1 (50) |
| back pain | 5 | 3 (60) |
| blurred vision | 5 | 2 (40) |
| chest pain | 1 | 1 (0) |
| cold feeling in the extremities | 14 | 6 (42.9) |
| constipation | 5 | 3 (60) |
| cough | 5 | 3 (60) |
| deafness | 2 | 1 (50) |
| diarrhea | 3 | 3 (100) |
| dizziness | 5 | 3 (60) |
| ear ringing | 7 | 1 (14.3) |
| enervation | 4 | 3 (75) |
| exanthema | 4 | 1 (25) |
| eyestrain | 5 | 1 (20) |
| facial edema | 1 | 1 (100) |
| facial numbness | 2 | 0 (0) |
| facial paralysis | 1 | 1 (100) |
| facial stiffness | 1 | 0 (0) |
| fatigue | 20 | 11 (55) |
| generalized muscle stiffness | 1 | 0 (0) |
| gingival pain | 1 | 0 (0) |
| glycosuria | 7 | 4 (57.1) |
| headache | 12 | 11 (91.7) |
| heavy feeling in the body | 4 | 2 (50) |
| heavy feeling in the head | 1 | 0 (0) |
| heavy feeling in the legs | 1 | 1 (100) |
| heavy stomach feeling | 1 | 0 (0) |
| hypertension | 10 | 4 (40) |
| insomnia | 17 | 8 (47.1) |
| jaundice | 1 | 1 (100) |
| joint pain | 45 | 30 (66.7) |
| loss of appetite | 1 | 0 (0) |
| loss of grip | 1 | 0 (0) |
| lower back pain | 89 | 51 (57.3) |
| menstrual irregularity | 1 | 0 (0) |
| pain in the extremities | 31 | 10 (32.3) |
| palpitation | 1 | 1 (100) |
| paralysis in the extremities | 3 | 0 (0) |
| plantar edema | 4 | 2 (50) |
| pollakiuria | 1 | 1 (100) |
| pruritus cutaneous | 10 | 4 (40) |
| rigidity of the arms | 1 | 1 (100) |
| sensation of numbness in the | 29 | 11 (38.0) |
| extremities | | |
| separation of the calx epidermis | 1 | 1 (100) |
| shoulder or neck pain | 25 | 14 (56) |
| shoulder or neck stiffness | 90 | 52 (57.8) |
| sore throat | 2 | 1 (50) |
| stomachache | 5 | 4 (80) |
| swelling of joints | 2 | 2 (100) |
| trembling of the extremities | 1 | 1 (100) |
| urinary incontinence | 1 | 0 (0) |
| total | 505 | 268 (53.1) |

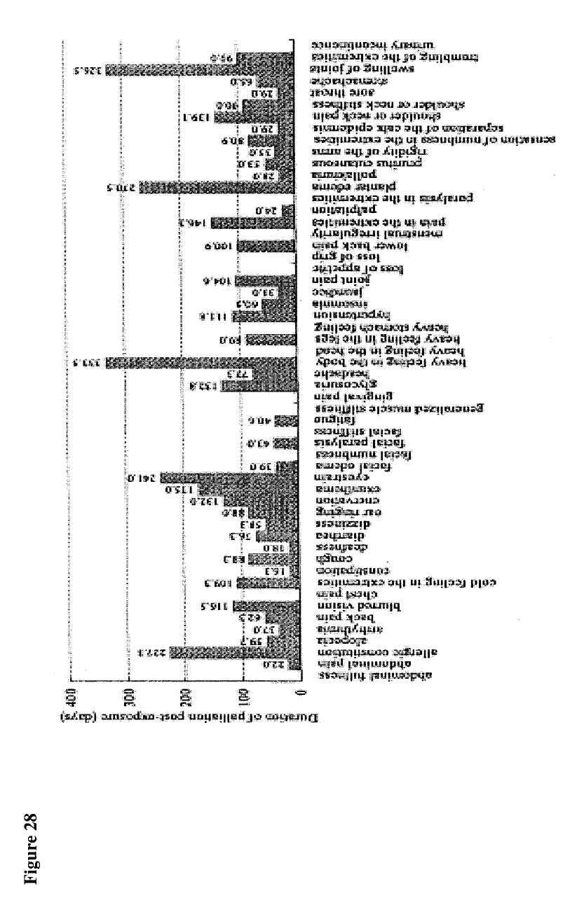

FIG. 28 shows mean duration of palliation per symptom irrespective of the frequency/interval of Healthtron therapy in 505 patients. Considering the small sample size in many of the symptoms identified, an inherent limitation in this study where the researchers were solely dependent on data generated from the questionnaire, we believe that the persistence of the palliative effect of therapy could be validly described only in those symptoms that were identified by at least 10 patients showing >50% palliation rate. Palliation of fatigue lasted for about 50 days; joint, lower back and shoulder/neck stiffness were palliated for a little less than 100 days. The longer mean duration of palliation noted among many other symptoms could be a reflection of the sample size rather than the real effect of therapy.

F. Method of Optimizing Electrical Therapy Parameters

The selection and control of parameter ranges of the invention enables the utilization of EF as a therapeutic tool, while avoiding unwanted side effects which may result from its use. Accordingly, the invention provides parameters and ranges of their use that enable a trained individual to use EF as a therapeutic tool to achieve a specific biological result and to avoid unwanted side effects.

A preferred method of determining optimal parameters for EF therapy includes the following steps: (i) identifying a desired biological response to elicit in a living organism; (ii) selecting or measuring a mean induced current density over membranes of cells in the organism or in a tissue sample or culture derived from the organism; (iii) selecting or measuring an external electric field that generates the selected or measured induced current density at a particular distance from the organism, sample or culture; (iv) selecting or measuring a continuous period of time to generate the selected or measured induced current density over the membranes; (v) applying the selected or measured electric field to the organism, sample or culture to generate the selected or measured induced current density over the cell membranes for the selected or measured continuous period of time; (vi) determining the extent to which the desired biological response occurs; (vii) optionally repeating any of steps (ii) through (vi); and (viii) identifying the values for the selected or measured induced current density, for the selected or measured external electric field, or for the selected or measured continuous period of time that optimally elicit the desired biological response.

Preferably, the method further includes, before step (viii), generating a dose-response curve as a function of either the selected or measured induced current density, the selected or measured external electric field, or the selected or measured continuous period of time. Still more preferably, the method further comprises, before step (viii), selecting or measuring the following: a number of times that step (v) is repeated, the interval of time between the repetitions of step (v), and the overall duration of time that the selected or measured induced current density is generated over the membranes.

More preferred embodiments include one or more of the following features: the selected or measured induced current density is about 0.001 mA/m2 to about 15 mA/m2; the induced current density is selected or measured by measuring the induced current flowing in a given section of the living organism or portion thereof, by converting the measured current into a voltage signal, by converting the voltage signal into an optical signal, by then reconverting the optical signal into a voltage signal, and analyzing the waveform and frequency; and/or the external electric field (E) is selected or measured in terms of the expression E=I/εoωS, where S is a section of the electric field measurement sensor, εo is an induction rate in a vacuum, I is a current, and εoωS is 2πf, and f is frequency.

A preferred method of determining optimal parameters for applied current therapy includes the following steps: (i) identifying a desired biological response to elicit in a living organism or portion thereof; (ii) selecting or measuring a mean applied current density over the membranes of cells in the organism or in a tissue sample or culture derived therefrom, wherein the mean applied current density is about 10 mA/m2 to about 2,000 mA/m2; (iii) selecting or measuring an electric current that will generate the selected or measured applied current density; (iv) selecting or measuring a continuous period of time to generate the selected or measured applied current density; (v) applying the selected or measured electric current to generate the selected or measured applied current density for the selected or measured continuous period of time; (vi) determining the extent to which the desired biological response occurs; (vii) repeating any of steps (ii) through (vi) to generate a dose-response curve as a function of the selected or measured electric current, the selected or measured applied current density, or the selected or measured continuous period of time; and (viii) identifying the values for the selected or measured electric current, for the selected or measured applied current density, or for the selected or measured continuous period of time that optimally elicit the desired biological response. Preferably, the method further includes, before step (viii), selecting or measuring the following: a number of times that step (v) is repeated, the interval of time between the repetitions of step (v), and the overall duration of time that the applied current density is generated over the membranes.

The inventors have determined parameters that optimally treat certain disorders. Broadly speaking, EF voltage (exogenous) may be applied in the range of between about 50 V to about 30 kV. Induced current density may be generated in the range of between about 0.001 to about 15 mA/m². Preferably, EF induced current density is generated in the range of between about 0.012 to about 11.1 mA/m², more preferably about 0.026 to about 5.55 mA/m².

Applied current density may be utilized in the range of between about 10 to about 2,000 mA/m². In another embodiment of the invention, applied current is generated in the range of between about 50 to about 600 mA/m². In a further embodiment of the invention, EF applied current is generated in the range of between about 60 to about 100 mA/m².

Table 11 provides preferred parameter sets for the treatment of disorders and conditions. Table 11 provides the particular disorder, condition, organ or system to which the parameter set is applied. Table 11 also provides the particular parameter values, although it is to be understood that the values are approximations and equivalent ranges are contemplated by the invention.

TABLE 11
Preferred Parameters

| | | EF | | Induced | Applied Current | |
|---|---|---|---|---|---|---|
| Parameter | Disorder, Condition, Organ or | Frequency | EF Voltage | Current Density | Density | Duration of |
| Set | System | (in Hertz) | (in volts) | (in mA/m²) | (in mA/m²) | Exposure |
| 1 | Disorders associated with | 60 | | | 60, 200, 600, or | 4 min |
| | cellular Ca²⁺ levels | | | | 2,000 | |
| 2 | Disorders associated with | 60 | | | 2000 | 2 min |
| | cellular Ca²⁺ levels | | | | | |
| 3 | Disorders associated with | 60 | | | 10, 50, and 100 | 24 hours/day |
| | fibroblast proliferation | | | | | for 7 days |
| 4 | Disorders associated with | 50 | | 0.42 | | 2 and 24 |
| | cellular Ca²⁺ levels | (30 kV/m) | | | | hours/day |
| 5 | Rheumatoid Arthritis | 50 | 2000 | 0.026-0.32 | | 2 hours/day for |
| | | | | | | 56 days |
| 6 | Disorders associated with | 50 | 3000 | 0.42 | | 24 hours |
| | cellular Ca²⁺ levels | (30 kV/m) | | | | |
| 7 | Disorders associated with | 60 | | | 60 or 600 | 30 min and 24 |
| | cellular Ca²⁺ levels | | | | | hours |
| 8 | Disorders associated with | 60 | | | 60 | 12 min |
| | cellular Ca²⁺ levels | | | | | |
| 9 | Disorders associated with | 60 | | | 60 | 4 min |
| | cellular Ca²⁺ levels | | | | | |
| 10 | Reduction in Stress Levels and | 50 | 7000 | 0.035-0.5 | | 60 min |
| | Associated Disorders | (17.5 | | | | |
| | | kV/m) | | | | |
| 11 | Disorders associated with | 60 | | | 60 | 12 min |
| | cellular Ca²⁺ levels | | | | | |
| 12 | Disorders associated with | 60 | | | 60 | 4 min |
| | cellular Ca²⁺ levels | | | | | |
| 13 | Disorders associated with | 50 | 3000 | 0.42 | | 24 hrs |
| | cellular Ca²⁺ levels | (30 kV/m) | | | | |
| 14 | Cellular Proliferative Disorders | 50 | | | 10, 50, and 100 | 7 days |
| 15 | Increase the Induction Response | 60 | | | 60 or 600 | 30 min and 24 |
| | of Immune System cells to ConA | | | | | hrs |
| 16 | Increase the Induction Response | 60 | | | 60 | 12 min |
| | of Immune System cells to ConA | | | | | |
| 17 | Disorders Associated with | | 50 and | 0.0001-0.42 | | 1 hr/day for 72 |
| | Electrolyte Imbalance | | 15000 (AC, | | | or 100 days |
| | | | DC+, DC−) | | | |
| 18 | Arthralgia, Severe Stress, | | 9000 or | 2.3-11.1 | | 7 times by 7000 |
| | Chronic Insomnia and Chronic | | 30000 | | | V; 23 times by |
| | Allergy | | | | | 30000 V |
| 19 | Fatigue | AC | 30000 | 7.5-11.1 | | 2 or 3 thirty |
| | | | | | | minute |
| | | | | | | sessions/week, |
| | | | | | | with a total of 5 |
| | | | | | | sessions per |
| | | | | | | patient, each |
| | | | | | | session lasting |
| | | | | | | 30 mins. |
| 20 | Stress response and Cytokine- | 40000 | 8000 | 0.08-1.12 | | 2 hours |
| | induced Disorders | | | | | |
| 21 | Disorders Associated with | AC | 15000 | 3.75-5.55 | | 30 min/session, |
| | Electrolyte Imbalance | | | | | every other day |
| | | | | | | for 14 days |
| 22 | Suppression of Body weight | 50 (12-40 | | 0.70-1.12 | | 30-120 min/day |
| | | kV/m) | | | | for 28 days |
| 23 | Cellular Proliferative Disorders | 50 (12-40 | | 0.024-1.12 | | 30-120 min/day |
| | | kV/m) | | | | for 56 days |

The invention is also directed to a method of determining a desired set of parameters such as EF characteristics, induced current density, applied current density, and duration of exposure, such that the maximum desired effect is obtained in the biological test subject.

In a preferred embodiment of the invention, the method of optimization involves the following steps: identification of a desired biological effect (e.g., cause an inward calcium ion flux in muscle cells) to elicit in an organism or portion thereof; selection of a value for a mean applied current density or for an induced current density at the cell membranes of the organism or portion thereof, wherein the value preferably falls within the range of about 10 mA/m² to about 2,000 mA/m² in the case of applied current and within the range of about 0.001 mA/m² to about 15 mA/m² in the case of induced current; determination of values (such as frequency and EF voltage) for the applied current or EF that will generate the selected current density; selecting a discrete period of time to generate the applied current density, wherein the period falls within the range of about 2 minutes to about 10,080 continuous or non-continuous minutes; application of the applied current or EF to generate the selected current density; determination of the extent to which the desired biological effect occurs; and repetition of any of the steps. Preferably, the optimization procedure also entails generation of a dose-response curve as a function of the selected values. In another preferred embodiment, the values for the applied current or EF are determined in view of the organism's body morphology, weight, percent body fat, and other factors relevant to induction of current over cell membranes.

In some embodiments of the invention, the parameters used for in vivo modulation of ion flux across cellular membranes are exemplified by the combinations presented in Table 12. In other embodiments of the invention, the parameters used for in vitro modulation of ion flux across cellular membranes are exemplified by the combinations presented in Table 13.

TABLE 12
Exemplary Parameters for in vivo Modulation of Ion Flux

| | | | Induced Current | Applied Current | |
|---|---|---|---|---|---|
| Parameter | EF voltage | EF frequency | Density | Density | Duration of |
| Set | (in volts) | (in Hz) | (in mA/m²) | (in mA/m²) | Exposure |
| 1 | 2,000 | 50 | 0.026-0.32 | | 2 hr/day for 7 |
| | | | | | days |
| 2 | 2,000 | 50 | 0.026-0.32 | | 2 hr/day for |
| | | | | | 56 days |
| 3 | 7,000 | 50 (17.5 | 0.035-0.5 | | 60 min. |
| | | KV/m) | | | |
| 4 | 30,000 | 60 | | 7.5-11.1 | 30 min. |
| 5 | 7,700 | 50 | 0.015-0.22 | | 2 hrs./day, 6 |
| | | | | | days/week, |
| | | | | | for 15 weeks |
| 6 | 15,000 | 60 | 3.8-5.6 | | 20 min./day, |
| | | | | | 4X per |
| | | | | | session for 15 |
| | | | | | days |
| 7 | 50 | 50 | 0.0001-0.42 | | 72 days |
| 8 | 15,000 | 50 | 0.0001-0.42 | | 100 days |
| 9 | 3,000 | 60 | 0.006-0.08 | | 35 days |
| 10 | 10,000 | 60 | 0.05-0.7 | | 15 min./day |
| | | | | | for 91 days |
| 11 | 7,000 | 60 (17.5 | 0.035-0.5 | | 15 min./day |
| | | KV/m) | | | for 7 days |
| 12 | 8,000 | 40 KV/m | | | 2 hrs. |
| 13 | 15,000 | 50 | 3.75-5.55 | | 30 |
| | | | | | min/session, |
| | | | | | every other |
| | | | | | day for 2 |
| | | | | | weeks |
| 14 | 10,000- | 50 | 2.5-11.1 | | 30 min. |
| | 30,000 | | | | |
| 15 | 30,000 | 50 | 7.5-11.1 | | 15 min./day, |
| | | | | | 3X/week for |
| | | | | | 2 weeks |
| 16 | 30,000 | 50 | 7.5-11.1 | | 30 min./day |
| 17 | 30,000 | 60 | 7.5-11.1 | | 30 min./day |
| 18 | 2,400 | 50 (6 KV/m) | 0.012-0.17 | | |
| 19 | 8,000 | 50 (40 KV/m) | 0.08-1.12 | | 2 hrs. |
| 20 | 1,200 | 50 (6 KV/m) | 0.012-0.17 | | 1 hr./day for |
| | | | | | 7 days |
| 21 | | 50 (12-40 | 0.024-1.12 | | 30-120 |
| | | KV/m) | | | min./day for |
| | | | | | 4 weeks |
| 22 | | 50 (12-40 | 0.024-1.12 | | 30-120 |
| | | KV/m) | | | min./day for |
| | | | | | 8 weeks |
| 23 | 2,400 | 50 (6 KV/m) | 0.012-0.17 | | 30 min. |
| 24 | 2,400 | 50 (6 KV/m) | 0.012-0.17 | | 120 min. |
| 25 | 10,000; | | 2.5-11.1 | | 20 min. |
| | 20,000; or | | | | |
| | 30,000 | | | | |
| 26 | 10,000 | | 2.5-3.7 | | 10 min./day, |
| | | | | | 3X/week for |
| | | | | | 5 weeks |

TABLE 13
Exemplary Parameters for in vitro Modulation of Ion Flux

| | | | Induced Current | Applied Current | |
|---|---|---|---|---|---|
| | EF voltage | EF frequency | Density | Density | Duration of |
| Parameter | (in volts) | (in Hz) | (in mA/m²) | (in mA/m²) | Exposure |
| 1 | | 60 | | 60 | 4 min. |
| 2 | | 60 | | 200 | 4 min. |
| 3 | | 60 | | 600 | 4 min. |
| 4 | | 60 | | 2000 | 4 min. |
| 5 | | 60 | | 2000 | 4 min. |
| 6 | | 60 | | 10 | 24 hr/day for |
| | | | | | 7 days |
| 7 | | 60 | | 50 | 24 hr/day for |
| | | | | | 7 days |
| 8 | | 60 | | 100 | 24 hr/day for |
| | | | | | 7 days |
| 9 | | 50 (30 KV/m) | 0.42 | | 2 hr |
| 10 | | 50 (30 KV/m) | 0.42 | | 24 hr |
| 11 | | 50 (30 KV/m) | 0.42 | | 24 hrs. |
| 12 | | 60 | | 60 or 600 | 30 min. |
| 13 | | 60 | | 60 or 600 | 24 hrs. |
| 14 | | 60 | | 60 | 12 min. |
| 15 | | 60 | | 60 | 4 min. |
| 16 | 3,000 | 50 (30 KV/m) | 0.42 | | 24 hrs. |
| 17 | | 50 | 100-1000 | | |
| 18 | | 50 | 10 | | 7 days |
| 19 | | 50 | 50 | | 7 days |
| 20 | | 50 | 100 | | 7 days |
| 21 | 15,000 | 60 | | | |
| 22 | 1,000 | 50 (150 | 3.9 | | 48 hrs. |
| | | KV/m) | | | |
| 23 | 1,000 | 50 (10 KV/m) | 0.26-0.34 | | 48 hrs. |
| 24 | | 50 (8.3 KV/m) | 0.28 | | 48 hrs. |

In an alternative embodiment, the invention is useful as a diagnostic tool to determine wether an individual is suffering from a particular disorder or condition. The specific parameters associated with the prevention, amelioration and treatment of a disorder or condition may be useful for detecting the presence of the same disorder or condition. The parameters can be applied as a diagnostic, and the effects monitored for responsiveness. If the patient is non-responsive to a given set of parameters associated with the disease, then the lack of a response suggests that the patient is not suffering from the particular disorder or condition. Alternatively, if the patient is responsive to a given set of parameters (associated with the disease), then the presence of a response is indicative of the presence of that particular disorder and/or condition. The diagnostic embodiments of the invention may be used for every disorder and/or condition for which a particular set of EF parameters has been determined.

It will be clear that the invention may be practiced otherwise than as particularly described in the foregoing description and examples. Numerous modifications and variations of the invention are possible in light of the above teachings and, therefore, are within the scope of the appended claims.

The entire disclosures of each document cited (including patents, patent applications, journal articles, abstracts, laboratory manuals, books, or other disclosures) in the Background of the invention, Detailed Description, and Examples are herein incorporated by reference in their entireties

Certain electric therapy apparatuses and methods of applying electric fields were disclosed in U.S. patent application Ser. No. 10/017,105, filed December 14, 2001, which is herein incorporated by reference in its entirety.

1. A method of treating or preventing a disorder that causes or is caused by an abnormal concentration of ions in cells of an organism or of a portion thereof, comprising restoring a normal concentration of ions to the cells, which includes applying to the organism or portion an external electric field that generates a mean induced current density of about 0.001 mA/m² to about 15 mA/m² over the membranes of the cells.

2. The method of claim 1, wherein the ions comprise calcium ions.

3. The method of claim 1, further comprising providing to the organism or portion thereof a calcium supplement, a vitamin D supplement, a lectin supplement, or a combination of said supplements.

4. The method of claim 3, wherein the lectin supplement is provided and the lectin supplement comprises concanavalin A or wheat germ agglutinin.

5. The method of any one of claims 1-4, wherein the mean induced current density is about 0.01 mA/m² to about 2 mA/m².

6. The method of claim 5, wherein the organism is a human and the electric field generates the mean induced current density over the membranes of the human's cells for a continuous period of about 10 minutes to about 240 minutes.

7. The method of claim 6, further comprising subsequently re-applying the external electric field to the human or portion thereof and re-generating the mean induced current density for additional continuous periods of about 30 minutes to about 90 minutes.

8. A device for carrying out the method of claim 1, wherein the device is an electric field therapy apparatus comprising:
(a) a main electrode and an opposed electrode;
(b) a voltage generator for applying a voltage to the electrodes;
(c) an induced current generator that controls the external electric field by varying the voltage or the distance between the opposed electrode and the organism or portion thereof; and
(d) a power source for driving the voltage generator.

9. The device of claim 8, wherein the main electrode does not contact the organism or portion thereof.

10. A method of treating a proliferative cell disorder comprising altering the flux of ions across cell membranes of an organism or a portion thereof, which includes applying to the organism or portion an external electric field that generates a mean induced current density of about 0.1 mA/m² to about 2 mA/m² over the cell membranes.

11. The method of claim 10, wherein the mean induced current density is about 0.2 mA/m² to about 1.2 mA/m².

12. The method of claim 11, wherein the mean induced current density is about 0.29 mA/m² to about 1.12 mA/m².

13. The method of claim 11, wherein the ions comprise calcium ions.

14. The method of claim 10, further comprising providing to the organism or portion thereof a calcium supplement, a vitamin D supplement, a lectin supplement, or a combination of said supplements.

15. The method of claim 14, wherein the lectin supplement is provided and the lectin supplement comprises concanavalin A or wheat germ agglutinin.

16. The method of claim 11, wherein the proliferative cell disorder involves differentiated fibroblast cells.

17. The method of claim 11 or 14, wherein the organism is a human and the electric field generates the mean induced current density over the membranes of the human's cells for a continuous period of about 10 minutes to about 240 minutes.

18. The method of claim 17, further comprising subsequently re-applying the electric field to the human or portion thereof and re-generating the mean induced current density for additional continuous periods of about 30 minutes to about 90 minutes.

19. The method of claim 18, wherein the human is disposed in a hospital or clinic bed.

20. A device for carrying out the method of claim 11, wherein the device is an electric field therapy apparatus comprising:
(a) a main electrode and an opposed electrode;
(b) a voltage generator for applying a voltage to the electrodes;
(c) an induced current generator that controls the external electric field by varying the voltage or the distance between the opposed electrode and the organism or portion thereof; and
(d) a power source for driving the voltage generator.

21. The device of claim 20, wherein the main electrode does not contact the organism or portion thereof.

22. A method of treating electrolyte imbalance comprising altering the flux of ions across cell membranes of an organism or portion thereof, which includes applying to the organism or portion an external electric field that generates a mean induced current density of about 0.4 mA/m² to about 6.0 mA/m² over the cell membranes.

23. The method of claim 22, wherein the mean induced current density is about 0.4 mA/m² to about 5.6 mA/m².

24. The method of claim 23, wherein the mean induced current density is about 0.43 mA/m² to about 5.55 mA/m².

25. The method of claim 23, wherein the ions comprise calcium ions.

26. The method of claim 22, further comprising providing to the organism or portion thereof a calcium supplement, a vitamin D supplement, a lectin supplement, or a combination of said supplements.

27. The method of claim 26, wherein the lectin supplement is provided and the lectin supplement comprises concanavalin A or wheat germ agglutinin.

28. The method of claim 23 or 26, wherein the organism is a human and the electric field generates the mean induced current density over the membranes of the human's cells for a continuous period of about 10 minutes to about 240 minutes.

29. The method of claim 28, further comprising subsequently re-applying the electric field to the human or portion thereof and re-generating the mean induced current density for additional continuous periods of about 30 minutes to about 90 minutes.

30. The method of claim 29, wherein the human is disposed in a hospital or clinic bed.

31. A device for carrying out the method of claim 22, wherein the device is an electric field therapy apparatus comprising:
(a) a main electrode and an opposed electrode;
(b) a voltage generator for applying a voltage to the electrodes;
(c) an induced current generator that controls the external electric field by varying the voltage or the distance between the opposed electrode and the organism or portion thereof; and
(d) a power source for driving the voltage generator.

32. The device of claim 31, wherein the main electrode does not contact the organism or portion thereof.

33. A method of treating disorders associated with serum calcium concentrations comprising altering the flux of calcium ions across cell membranes of an organism or portion thereof, which includes applying to the organism or portion an external electric field that generates a mean induced current density of about 0.3 mA/m² to about 0.6 mA/m² over the cell membranes.

34. The method of claim 33, wherein the mean induced current density is about 0.4 mA/m² to about 0.5 mA/m².

35. The method of claim 34, wherein the mean induced current density is about 0.42 mA/m².

36. The method of claim 33, further comprising providing to the organism or portion thereof a calcium supplement, a vitamin D supplement, a lectin supplement, or a combination of said supplements.

37. The method of claim 36, wherein the lectin supplement is provided and the lectin supplement comprises concanavalin A or wheat germ agglutinin.

38. The method of claim 34 or 36, wherein the organism is a human and the electric field generates the mean induced current density over the membranes of the human's cells for a continuous period of about 10 minutes to about 240 minutes.

39. The method of claim 38, further comprising subsequently re-applying the electric field to the human or portion thereof and re-generating the mean induced current density for additional continuous periods of about 30 minutes to about 90 minutes.

40. The method of claim 39, wherein the human is disposed in a hospital or clinic bed.

41. A device for carrying out the method of claim 33, wherein the device is an electric field therapy apparatus comprising:
(a) a main electrode and an opposed electrode;
(b) a voltage generator for applying a voltage to the electrodes;
(c) an induced current generator that controls the external electric field by varying the voltage or the distance between the opposed electrode and the organism or portion thereof; and
(d) a power source for driving the voltage generator.

42. The device of claim 41, wherein the main electrode does not contact the organism or portion thereof.

43. A method of reducing levels of ACTH or cortisol, comprising altering the flux of ions across cell membranes of an organism or portion thereof, which includes applying to the organism or portion an external electric field that generates a mean induced current density of about 0.03 mA/m² to about 12 mA/m² over the cell membranes.

44. The method of claim 43, wherein the mean induced current density is about 0.035 mA/m² to about 11.1 mA/m².

45. The method of claim 44, wherein the mean induced current density is about 0.035 to about 0.5 mA/m².

46. The method of claim 43, wherein the ions comprise calcium ions and the method further comprises providing to the organism or portion thereof a calcium supplement, a vitamin D supplement, a lectin supplement, or a combination of said supplements.

47. The method of claim 46, wherein the lectin supplement is provided and the lectin supplement comprises concanavalin A or wheat germ agglutinin.

48. The method of claim 44 or 46, wherein the organism is a human and the electric field generates the mean induced current density over the membranes of the human's cells for a continuous period of about 10 minutes to about 240 minutes.

49. The method of claim 48, further comprising subsequently re-applying the electric field to the human or portion thereof and re-generating the mean induced current density for additional continuous periods of about 30 minutes to about 90 minutes.

50. The method of claim 49, wherein the human is disposed in a hospital or clinic bed.

51. A device for carrying out the method of claim 43, wherein the device is an electric field therapy apparatus comprising:
(a) a main electrode and an opposed electrode;
(b) a voltage generator for applying a voltage to the electrodes;
(c) an induced current generator that controls the external electric field by varying the voltage or the distance between the opposed electrode and the organism or portion thereof; and
(d) a power source for driving the voltage generator.

52. The device of claim 51, wherein the main electrode does not contact the organism or portion thereof.

53. A method of treating stress comprising altering the flux of ions across cell membranes of an organism or portion thereof, which includes applying to the organism or portion an external electric field that generates a mean induced current density of about 0.03 mA/m² to about 12 mA/m² over the cell membranes.

54. The method of claim 53, wherein the mean induced current density is about 0.035 mA/m² to about 11.1 mA/m².

55. The method of claim 54, wherein the ions comprise calcium ions.

56. The method of claim 53, further comprising providing to the organism or portion thereof a calcium supplement, a vitamin D supplement, a lectin supplement, or a combination of said supplements.

57. The method of claim 56, wherein the lectin supplement is provided and the lectin supplement comprises concanavalin A or wheat germ agglutinin.

58. The method of claim 54 or 56, wherein the organism is a human and the electric field generates the mean induced current density over the membranes of the human's cells for a continuous period of about 10 minutes to about 240 minutes.

59. The method of claim 58, further comprising subsequently re-applying the electric field to the human or portion thereof and re-generating the mean induced current density for additional continuous periods of about 30 minutes to about 90 minutes.

60. The method of claim 59, wherein the human is disposed in a hospital or clinic bed.

61. A device for carrying out the method of claim 53, wherein the device is an electric field therapy apparatus comprising:
(a) a main electrode and an opposed electrode;
(b) a voltage generator for applying a voltage to the electrodes;
(c) an induced current generator that controls the external electric field by varying the voltage or the distance between the opposed electrode and the organism or portion thereof; and
(d) a power source for driving the voltage generator.

62. The device of claim 61, wherein the main electrode does not contact the organism or portion thereof.

63. A method of treating arthritis comprising altering the flux of ions across cell membranes of an organism or portion thereof, which includes applying to the organism or portion an external electric field that generates a mean induced current density of about 0.02 mA/m² to about 0.4 mA/m² over the cell membranes.

64. The method of claim 63, wherein the mean induced current density is about 0.025 mA/m² to about 0.35 mA/m².

65. The method of claim 64, wherein the mean induced current density is about 0.026 mA/m² to about 0.32 mA/m².

66. The method of claim 64, wherein the ions comprise calcium ions.

67. The method of claim 63, further comprising providing to the organism or portion thereof a calcium supplement, a vitamin D supplement, a lectin supplement, or a combination of said supplements.

68. The method of claim 67, wherein the lectin supplement is provided and the lectin supplement comprises concanavalin A or wheat germ agglutinin.

69. The method of claim 64 or 67, wherein the organism is a human and the electric field generates the mean induced current density over the membranes of the human's cells for a continuous period of about 10 minutes to about 240 minutes.

70. The method of claim 69, further comprising subsequently re-applying the electric field to the human or portion thereof and re-generating the mean induced current density for additional continuous periods of about 30 minutes to about 90 minutes.

71. The method of claim 70, wherein the human is disposed in a hospital or clinic bed.

72. A device for carrying out the method of claim 63, wherein the device is an electric field therapy apparatus comprising:
(a) a main electrode and an opposed electrode;
(b) a voltage generator for applying a voltage to the electrodes;
(c) an induced current generator that controls the external electric field by varying the voltage or the distance between the opposed electrode and the organism or portion thereof; and
(d) a power source for driving the voltage generator.

73. The device of claim 72, wherein the main electrode does not contact the organism or portion thereof

74. A method of treating excessive body weight comprising altering the flux of ions across cell membranes of an organism or portion thereof, which includes applying to the organism or portion an external electric field that generates a mean induced current density of about 0.02 mA/m² to about 1.5 mA/m² over the cell membranes.

75. The method of claim 74, wherein the mean induced current density is about 0.02 mA/m² to about 1.2 mA/m².

76. The method of claim 75, wherein the mean induced current density is about 0.024 mA/m² to about 1.12 mA/m².

77. The method of claim 75, wherein the ions comprise calcium ions.

78. The method of claim 74, further comprising providing to the organism or portion thereof a calcium supplement, a vitamin D supplement, a lectin supplement, or a combination of said supplements.

79. The method of claim 78, wherein the lectin supplement is provided and the lectin supplement comprises concanavalin A or wheat germ agglutinin.

80. The method of claim 74 or 78, wherein the organism is a human and the electric field generates the mean induced current density over the membranes of the human's cells for a continuous period of about 10 minutes to about 240 minutes.

81. The method of claim 80, further comprising subsequently re-applying the electric field to the human or portion thereof and re-generating the mean induced current density for additional continuous periods of about 30 minutes to about 90 minutes.

82. The method of claim 81, wherein the human is disposed in a hospital or clinic bed.

83. A device for carrying out the method of claim 73, wherein the device is an electric field therapy apparatus comprising:
(a) a main electrode and an opposed electrode;
(b) a voltage generator for applying a voltage to the electrodes;
(c) an induced current generator that controls the external electric field by varying the voltage or the distance between the opposed electrode and the organism or portion thereof; and
(d) a power source for driving the voltage generator.

84. The device of claim 83, wherein the main electrode does not contact the organism or portion thereof.

85. A method of determining optimum parameters of external electric field exposure for the treatment of a disorder, comprising:
(i) identifying a desired biological response to elicit in a living organism;
(ii) selecting or measuring a mean induced current density over membranes of cells in the organism or in a tissue sample or culture derived from the organism;
(iii) selecting or measuring an external electric field that generates the selected or measured induced current density at a particular distance from the organism, sample or culture;
(iv) selecting or measuring a continuous period of time to generate the selected or measured induced current density over the membranes;
(v) applying the selected or measured electric field to the organism, sample or culture to generate the selected or measured induced current density over the cell membranes for the selected or measured continuous period of time;
(vi) determining the extent to which the desired biological response occurs;
(vii) optionally repeating any of steps (ii) through (vi); and
(viii) identifying the values for the selected or measured induced current density, for the selected or measured external electric field, or for the selected or measured continuous period of time that optimally elicit the desired biological response.

86. The method of claim 85, further comprising, before step (viii), generating a dose-response curve as a function of the selected or measured induced current density, the selected or measured external electric field, or the selected or measured continuous period of time.

87. The method of claim 85, further comprising, before step (viii), selecting or measuring the following:
a number of times that step (v) is repeated,
the interval of time between the repetitions of step (v), and
the overall duration of time that the selected or measured induced current density is generated over the membranes.

88. The method of claim 85, wherein the selected or measured induced current density is about 0.001 mA/m² to about 15 mA/m².

89. The method of claim 85, wherein the cells are in a culture.

90. The method of claim 89, wherein the cells in culture are human cells.

91. The method of claim 85, wherein the cells are in a living organism or portion thereof.

92. The method of claim 91, wherein the living organism is a human.

93. The method of claim 85, wherein the induced current density is selected or measured by measuring the induced current flowing in a given section of the living organism or portion thereof, by converting the measured current into a voltage signal, by converting the voltage signal into an optical signal, by then reconverting the optical signal into a voltage signal, and analyzing the waveform and frequency.

94. The method of claim 85, wherein the induced current density is represented by J and J is expressed in terms of J=I/B.

95. The method of claim 85, further comprising providing to the organism, sample or culture a calcium supplement, a vitamin D supplement, a lectin supplement, or a combination of said supplements.

96. The method of claim 95, wherein the lectin supplement is provided and the lectin supplement comprises concanavalin A or wheat germ agglutinin.

97. A device for carrying out the method of claim 85, wherein the device is an electric field therapy apparatus comprising:
(a) a main electrode and an opposed electrode;
(b) a voltage generator for applying a voltage to the electrodes;
(c) an induced current generator that controls the external electric field by varying the voltage or the distance between the opposed electrode and the organism or portion thereof; and
(d) a power source for driving the voltage generator.

98. The device of claim 97, wherein the main electrode does not contact the organism or portion thereof.

99. A method of treating a proliferative cell disorder comprising altering the flux of ions across cell membranes of an organism or portion thereof, which includes contacting the organism or portion with an electric current that generates a mean applied current density of about 10 mA/m² to about 100 mA/m² over the cell membranes.

100. The method of claim 99, wherein the ions comprise calcium ions and the mean applied current density is generated over the cell membranes for a substantially continuous period of at least about 7 days.

101. The method of claim 99, further comprising providing to the organism or portion thereof a calcium supplement, a vitamin D supplement, a lectin supplement, or a combination of said supplements.

102. The method of claim 101, wherein the lectin supplement is provided and the lectin supplement comprises concanavalin A or wheat germ agglutinin.

103. The method of claim 99, 100 or 101, wherein the organism is a human.

104. An electric current therapy device for carrying out the method of claim 99.

105. A method of treating stress-related disorders or symptoms comprising altering the flux of ions across cell membranes of an organism or portion thereof, which includes contacting the organism or portion with an electric current that generates a mean applied current density of about 60 mA/m² to about 600 mA/m² over the cell membranes.

106. The method of claim 105, wherein the ions comprise calcium ions.

107. The method of claim 105, further comprising providing to the organism or portion thereof a calcium supplement, a vitamin D supplement, a lectin supplement, or a combination of said supplements.

108. The method of claim 107, wherein the lectin supplement is provided and the lectin supplement comprises concanavalin A or wheat germ agglutinin.

109. The method of claim 105 or 107, wherein the organism is a human.

110. An electric current therapy device for carrying out the method of claim 105.

111. A method of treating a disorder associated with serum calcium concentration comprising altering the flux of calcium ions across cell membranes of an organism or portion thereof, which includes contacting the organism or portion with an electric current that generates a mean applied current density of about 60 mA/m² to about 2,000 mA/m² over the cell membranes.

112. The method of claim 111, wherein the ions comprise calcium ions.

113. The method of claim 111, further comprising providing to the organism or portion thereof a calcium supplement, a vitamin D supplement, a lectin supplement, or a combination of said supplements.

114. The method of claim 113, wherein the lectin supplement is provided and the lectin supplement comprises concanavalin A or wheat germ agglutinin.

115. The method of claim 111 or 113, wherein the organism is a human.

116. The method of claim 111, wherein the current generates the mean applied current density over the cell membranes for a continuous period of about 1 minute to about 20 minutes.

117. The method of claim 116, wherein the current generates the mean applied current density over the cell membranes for a continuous period of about 2 minutes to about 10 minutes.

118. An electric current therapy device for carrying out the method of claim 111.

119. A method of determining optimum parameters of electric current exposure for the treatment of a disorder, comprising:
(i) identifying a desired biological response to elicit in a living organism or portion thereof;
(ii) selecting or measuring a mean applied current density over the membranes of cells in the organism or in a tissue sample or culture derived therefrom, wherein the mean applied current density is about 10 mA/m² to about 2,000 mA/m²;
(iii) selecting or measuring an electric current that will generate the selected or measured applied current density;
(iv) selecting or measuring a continuous period of time to generate the selected or measured applied current density;
(v) applying the selected or measured electric current to generate the selected or measured applied current density for the selected or measured continuous period of time;
(vi) determining the extent to which the desired biological response occurs;
(vii) repeating any of steps (ii) through (vi) to generate a dose-response curve as a function of the selected or measured electric current, the selected or measured applied current density, or the selected or measured continuous period of time; and
(viii) identifying the values for the selected or measured electric current, for the selected or measured applied current density, or for the selected or measured continuous period of time that optimally elicit the desired biological response.

120. The method of claim 119, further comprising, before step (viii), selecting or measuring the following:
a number of times that step (v) is repeated,
the interval of time between the repetitions of step (v), and
the overall duration of time that the applied current density is generated over the membranes.

121. The method of claim 120, wherein the cells are in a culture.

122. The method of claim 121, wherein the cells in culture are human cells.

123. The method of claim 120, wherein the cells are in a living organism or portion thereof.

124. The method of claim 123, wherein the living organism is a human.

125. The method of claim 120, further comprising providing to the organism, sample or culture a calcium supplement, a vitamin D supplement, a lectin supplement, or a combination of said supplements.

126. The method of claim 125, wherein the lectin supplement is provided and the lectin supplement comprises concanavalin A or wheat germ agglutinin.

127. An electric current therapy device for carrying out the method of claim 120.
